(19) United States
(12) Patent Application Publication
(10) Pub. No.: US 2012/0237472 A1
(43) Pub. Date: Sep. 20, 2012

(54) METHODS AND COMPOSITIONS FOR TREATING OR PREVENTING AUTOIMMUNE DISEASES USING IMMUNOMODULATORY AGENTS

(75) Inventors: Adam Ian Kaplin, Baltimore, MD (US); Mark T. Worthington, Prescott, AZ (US)

(73) Assignee: THE JOHNS HOPKINS UNIVERSITY, Baltimore, MD (US)

(21) Appl. No.: 13/386,730

(22) PCT Filed: Jul. 23, 2010
(86) PCT No.: PCT/US2010/043083

§ 371 Date: May 10, 2012

Related U.S. Application Data
(60) Provisional application No. 61/228,187, filed on Jul. 24, 2009.
(60) Provisional application No. 61/251,087, filed on Oct. 13, 2009.
(60) Provisional application No. 61/251,097, filed on Oct. 13, 2009.

(51) Int. Cl. A61K 38/02; A61K 38/07; A61K 38/10; A61K 38/17; A61K 38/08; A61K 38/19; A61P 37/06; A61P 37/02; A61P 1/00; A61P 29/00; A61P 19/02; A61P 3/10; A61P 3/00; A61P 5/14; A61P 1/16; A61P 5/38; A61K 39/395

(52) U.S. Cl. 424/85.1; 424/278.1; 424/173.1; 424/133.1; 424/142.1

(57) ABSTRACT

The present invention provides compositions and methods for treating and/or preventing an autoimmune disease comprising administering to a subject in need thereof an effective amount of cyclophosphamide and/or a cyclophosphamide derivative in combination with an additional immunomodulatory agent.

### RELATED APPLICATIONS

This application claims the benefit of U.S. Provisional Application No. 61/228,187, filed Jul. 24, 2009, U.S. Provisional Application No. 61/251,087, filed Oct. 13, 2009 and U.S. Provisional Application No. 61/251,097, filed Oct. 13, 2009, the contents of each of which are specifically incorporated by reference herein in their entirety.

### BACKGROUND

Autoimmune diseases are diseases characterized by a harmful immune response against an individual's own cells or tissues. Autoimmune diseases can be systemic or can affect a specific organ or tissue. For example, certain autoimmune diseases, such as multiple sclerosis, are related to immune responses against components of an individual's nervous system, while others, such as Crohn's disease, are related to immune responses against components of an individual's gastrointestinal tract.

Current therapies for many autoimmune diseases are either inefficacious or result in severe side-effects. For example, traditional treatments for Crohn's disease often include one or more surgical resections of the bowel, which can lead to a syndrome called “short gut syndrome,” where the remaining length of the bowel is insufficient to support life without lifetime intravenous feeding with total parenteral nutrition. Thus, there exists a need for improved compositions and methods for treating and/or preventing autoimmune diseases, including inflammatory bowel diseases such as Crohn's disease and neurological autoimmune diseases such as Multiple Sclerosis.

### SUMMARY

In some embodiments, the invention relates to methods for treating or preventing an autoimmune disease comprising administering to a subject in need thereof an effective amount of cyclophosphamide and/or a cyclophosphamide derivative and an additional immunomodulatory agent, wherein the additional immunomodulatory agent is selected from the group consisting of an amino acid co-polymer, a peptide fragment of myelin basic protein, an anti-tumor necrosis factor agent and pharmaceutically acceptable salts thereof.

In some embodiments, the invention relates to methods for treating or preventing an autoimmune disease comprising administering to a subject in need thereof an effective amount of cyclophosphamide and/or a cyclophosphamide derivative and an additional immunomodulatory agent, wherein the autoimmune disease is inflammatory bowel disease (e.g., ulcerative colitis or Crohn's disease), rheumatoid arthritis, diabetes mellitus, celiac disease, autoimmune thyroid disease, autoimmune liver disease, Addison's Disease, Sjögren's Syndrome, transplant rejection, graft vs. host disease, or host vs. graft disease, and the additional immunomodulatory agent is selected from the group consisting of an amino acid co-polymer, a peptide fragment of myelin basic protein, an anti-tumor necrosis factor agent and pharmaceutically acceptable salts thereof.

In some embodiments, the invention relates to methods for treating or preventing an autoimmune disease, the method comprising administering to a subject in need thereof an effective amount of cyclophosphamide and/or a cyclophosphamide derivative and an additional immunomodulatory agent, wherein the autoimmune disease is multiple sclerosis, Guillain-Barre syndrome, Lambert-Eaton myasthenic syndrome, myasthenia gravis, transverse myelitis, systemic lupus erythematosus (SLE or lupus), acute disseminated encephalomyelitis, autoimmune inner ear disease, narcolepsy, neuromyotonia, or schizophrenia; and the additional immunomodulatory agent is selected from the group consisting of an amino acid co-polymer other than glatiramir, a peptide fragment of myelin basic protein, an anti-tumor necrosis factor agent and pharmaceutically acceptable salts thereof.

In some embodiments, the invention relates to methods for preventing an autoimmune disease, the method comprising administering to a subject in need thereof an effective amount of an immunomodulatory agent, wherein the autoimmune disease is inflammatory bowel disease (e.g., ulcerative colitis or Crohn's disease), rheumatoid arthritis, diabetes mellitus, celiac disease, autoimmune thyroid disease, autoimmune liver disease, Addison's Disease, Sjögren's Syndrome, transplant rejection, graft vs. host disease, or host vs. graft disease, and the additional immunomodulatory agent is selected from the group consisting of an amino acid co-polymer, a peptide fragment of myelin basic protein, an anti-tumor necrosis factor agent and pharmaceutically acceptable salts thereof.

In some embodiments, the invention relates to compositions comprising (a) an effective amount of cyclophosphamide and/or a cyclophosphamide derivative and (b) an anti-lymphocyte antibody. In yet another embodiment, the invention provides a kit comprising compositions of the invention and instructions for use.

### DETAILED DESCRIPTION

### Definitions

As used herein, an “effective amount” is an amount effective for treating or preventing an autoimmune disease.

As used herein, the term “about” when used in conjunction with an immediately following numeric indication means the referenced numeric indication plus or minus up to 10% of that referenced numeric indication.

As used herein, the term “antibody” includes full-length antibodies and any antigen binding fragment (i.e., “antigen-binding portion”) or single chain thereof. The term “antibody” includes, but is not limited to, a glycoprotein comprising at least two heavy (H) chains and two light (L) chains inter-connected by disulfide bonds, or an antigen binding portion thereof. Antibodies may be polyclonal or monoclonal; xenogeneic, allogeneic, or syngeneic; or modified forms thereof (e.g., humanized, chimeric).

As used herein, the term “immunomodulatory agent” means a substance that has an effect on a subject's immune system.

As used herein, the term “treating” a disease in a subject or “treating” a subject having or suspected of having a disease refers to subjecting the subject to a pharmaceutical treatment, e.g., the administration of one or more agents, such that at least one symptom of the disease is decreased or prevented from worsening.

As used herein, the term “subject” refers to a mammal, e.g., a human, mouse, rat, guinea pig, dog, cat, horse, cow, pig, or non-human primate, such as a monkey, chimpanzee, baboon or rhesus. In one embodiment, the subject is a human.

### General

Certain embodiments of the invention relate to methods and compositions for treating and/or preventing an autoimmune disease. Some methods of the invention comprise administering to a subject, e.g., a subject in need thereof, an effective amount of (1) cyclophosphamide and/or a cyclophosphamide derivative and (2) an additional immunomodulatory agent, wherein the additional immunomodulatory agent is an amino acid co-polymer or a pharmaceutically acceptable salt thereof, a peptide fragment of myelin basic protein or a pharmaceutically acceptable salt thereof, or an anti-tumor necrosis factor agent.

In certain embodiments, the autoimmune disease is selected from a group consisting of inflammatory bowel disease (e.g., ulcerative colitis or Crohn's disease), rheumatoid arthritis, diabetes mellitus, celiac disease, autoimmune thyroid disease, autoimmune liver disease, Addison's Disease, Sjögren's Syndrome, transplant rejection, graft vs. host disease and host vs. graft disease. In certain embodiments, the autoimmune disease is a neurological autoimmune disease, such as multiple sclerosis. In certain embodiments the autoimmune disease is an inflammatory bowel disease, such as uncreative colitis or Crohn's disease.

### Neurological Autoimmune Diseases

Certain embodiments of the invention relate to methods or compositions for treating and/or preventing neurological autoimmune disease. Examples of neurological autoimmune diseases include, for example, multiple sclerosis, Guillain-Barre syndrome, Lambert-Eaton myasthenic syndrome, myasthenia gravis, transverse myelitis, systemic lupus erythematosus (SLE or lupus), acute disseminated encephalomyelitis, vasculitis, Sjogren's syndrome, Graves' disease, autoimmune inner ear disease, narcolepsy, neuromyotonia, and schizophrenia.

In some embodiments, the neurological autoimmune disease is multiple sclerosis (“MS”). MS is an autoimmune disease often characterized by recurrent episodes of demyelination and inflammation within the central nervous system. Symptoms of MS can include changes in sensation (e.g. hypoesthesias and paraesthesias), muscle weakness, muscle spasms, difficulty moving; difficulty with coordination and/or balance (e.g. ataxia); difficulty speaking (e.g. dysarthria), difficulty swallowing (e.g. dysphagia), difficulty controlling eye movement (e.g. nystagmus), impaired vision (e.g. diplopia), fatigue, pain (e.g. acute or chronic), difficulty controlling bladder function, difficulty controlling bowel function, and/or depression.

In some instances, MS can present as relapsing-remitting multiple sclerosis (RRMS). RRMS is typically characterized by periods of relapse/exacerbation (e.g. the unprovoked and unanticipated occurrence of a new symptom, or recurrence of an old symptom, lasting for a period of greater than 24 hours) followed by one or more periods of remission (e.g. periods with limited or no MS symptoms). In some individuals diagnosed with RRMS, the disorder progresses to secondary progressive MS. Secondary progressive MS (SPMS) is typically characterized by an initial period of relapsing-remitting MS, followed by progressive neurologic decline between relapses without any definite periods of remission. In some embodiments, the multiple sclerosis has relapsed. In some embodiments, the multiple sclerosis is in remission. In some embodiments, the multiple sclerosis in a progressive phase.

MS can be diagnosed, for example, using the McDonald Criteria. Table 1 sets forth data that can be used to diagnose MS based on an individual's clinical presentation.

TABLE 1

| Clinical Presentation | Additional Data |
|---|---|
| 2 or more attacks | None; clinical evidence will typically suffice |
| (relapses) | (additional evidence desirable but must be |
| 2 or more objective | consistent with MS) |
| clinical lesions | |
| 2 or more attacks | Dissemination in space, demonstrated by: |
| 1 objective clinical | MRI |
| lesion | or a positive CSF and 2 or more |
| | MRI lesions consistent with MS |
| | or further clinical attack involving |
| | different site |
| 1 attack | Dissemination in time, demonstrated by: |
| 2 or more objective | MRI |
| clinical lesions | or second clinical attack |
| 1 attack | Dissemination in space by demonstrated |
| 1 objective clinical | by: |
| lesion | MRI |
| (monosymptomatic | or positive CSF and 2 or more MRI |
| presentation) | lesions consistent with MS |
| | and |
| | Dissemination in time demonstrated by: |
| | MRI |
| | or second clinical attack |
| Insidious neurological | Positive CSF |
| progression suggestive of | and |
| MS (primary progressive | Dissemination in space demonstrated by: |
| MS) | MRI evidence of 9 or more T2 |
| | brain lesions |
| | or 2 or more spinal cord lesions |
| | or 4-8 brain and 1 spinal cord lesion |
| | or positive VEP with 4-8 MRI |
| | lesions |
| | or positive VEP with <4 brain |
| | lesions plus 1 spinal cord lesion |
| | and |
| | Dissemination in time demonstrated by: |
| | MRI |
| | or continued progression for 1 year |

Another clinical measure of disability progression and symptom severity is the Expanded Disability Status Scale (also known as “EDSS”). A commonly used clinical rating scale, the EDSS ranges from 0 (normal) to 10 (death due to MS), based on neurological examination of eight functional systems (visual, brainstem, sensory, cerebellar, sphincter, cerebral and others). EDSS can be a useful tool for classifying MS individuals by disease severity. EDSS measures impairment and disability based on the ratings of an observer or neurologist through a structured interview.

An additional measure of disability progression and symptom severity is the Multiple Sclerosis Functional Composite (“MSFC”). This scale is based on a composite score of three individual tests designed to test gait, upper extremity dexterity and cognition. The three subtests are: a) 25 foot timed walk (25TW); b)-9-hole peg test (9-HPT); and c) Paced Auditory Serial Addition Test (PASAT-3). The PASAT test requires individuals to add consecutive numbers as they are presented on an auditory tape and respond orally with the accurate sum. As each digit is presented, the individual must sum that number with the digit that was presented prior to it rather than with the individual's previous response.

### Inflammatory Bowel Disease

Certain embodiments of the invention relate to methods or compositions for treating and/or preventing neurological autoimmune disease.

As used herein, the term “inflammatory bowel diseases” or “IBD” includes art-recognized forms of a group of related conditions. Several major forms of IBD are known, and Crohn's disease (regional bowel disease, e.g., inactive and active forms) and ulcerative colitis (e.g., inactive and active forms) are the most common of these disorders. In addition, the IBD encompasses irritable bowel syndrome, microscopic colitis, lymphocytic-plasmocytic enteritis, coeliac disease, collagenous colitis, lymphocytic colitis and eosinophilic enterocolitis.

Other less common forms of IBD include, for example, indeterminate colitis, infectious colitis (viral, bacterial or protozoan, e.g. amoebic colitis) (e.g., clostridium dificile colitis), pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

### Achieving Immune Lymphablation

Cyclophosphamide and cyclophosphamide derivatives are lymphablative agents, and thus can be administered at an amount effective to achieve immune lymphablation. Immune lymphablation is achieved when the subject's white blood cell (“WBC”) count is about zero to about 100. Methods for measuring WBC in a subject are well known to those skilled in the art.

In some embodiments, the cyclophosphamide or cyclophosphamide derivative is administered at an amount of about 25 to about 75 mg/kg/day. In other embodiments, the cyclophosphamide or cyclophosphamide derivative is administered at an amount of about 50 mg/kg/day. In some embodiments, the cyclophosphamide or cyclophosphamide derivative is administered daily for a period of about 3 to 6 days, e.g., about 3, about 4, about 5, and/or to about 6 days. In other embodiments, the cyclophosphamide or cyclophosphamide derivative is administered daily for a period of about 4 days. In still other embodiments, the cyclophosphamide or cyclophosphamide derivative is administered daily for a period of about 4 days at an amount of about 50 mg/kg/day.

In some embodiments, the cyclophosphamide or cyclophosphamide derivative is administered in the form of a suspension or solution. In some embodiments, the cyclophosphamide solution comprises cyclophosphamide reconstituted from lyophilized cyclophosphamide. The lyophilized cyclophosphamide can be reconstituted, for example, in phosphate buffered saline (“PBS”), a saline solution, water, or any combination thereof. In some embodiments, the concentration of the cyclophosphamide in the solution is about 20 mg/mL. In one embodiment, the cyclophosphamide solution is administered intravenously.

In one embodiment, the cyclophosphamide derivative is 4-hydroperoxycyclophosphamide.

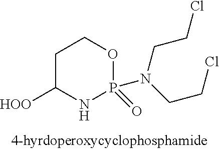

The synthesis of 4-hydroperoxycyclophosphamide is described, for example, in H-J. Hohorst, et al., “Synthesis of 4-Hydroperoxy Derivatives of Ifosamide and Trofosfamide by Direct Ozonation and Preliminary Antitumor Evaluation in Vivo,” Cancer Res., 36: 2278-2281 (1976) and G. Peter, et al., “Studies on 4-Hydroperoxycyclophosphamide (NSC-181815): A Simple Preparation Method and its Application for the Synthesis of a New Class of “Activated” Sulfur-Containing Cyclophosphamide (NSC-26271) Derivatives,” Cancer Treatment Reports, 60(4): 429-435 (1976), the contents of each of which are incorporated by reference herein in their entirety.

In another embodiment, the cyclophosphamide derivative is mafosfamide.

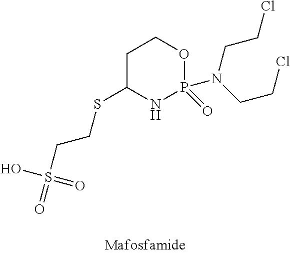

In another embodiment, the cyclophosphamide derivative is 4-hydroxycyclophosphamide, aldophosphamide, aldophosphamide hydrate, or iminophosphamide.

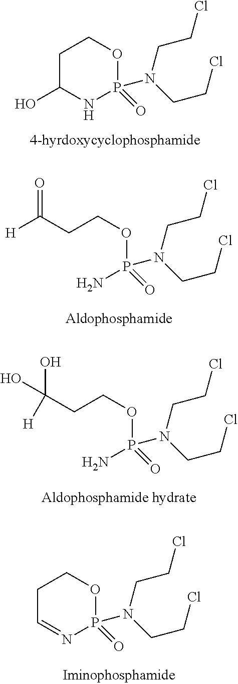

4-Hydroxycyclophosphamide and aldophosphamide are described in J. Zhang, et al., “Insights into oxazaphosphorine resistance and possible approaches to its circumvention,” Drug Resistance Updates, 8: 271-297 (2005), the contents of which are incorporated by reference herein in their entirety. 4-hydroxycyclophosphamide, aldophosphamide, aldophosphamide hydrate and iminophosphamide are also described in S. M. Ludeman, “The Chemistry of the Metabolites of Cyclophosphamide,” Current Pharm. Design, 5:627-43 (1999), the contents of which are incorporated by reference herein in their entirety.

In another embodiment, the cyclophosphamide derivative is 4-peroxycyclophosphamide, or a 4-(S-alkyl)cyclophosphamide.

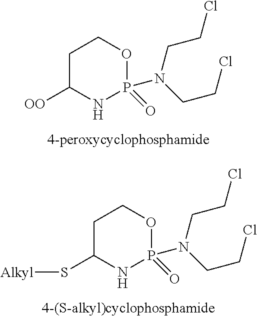

In one embodiment, the alkyl of the 4-(S-alkyl)cyclophosphamide is a C₁ to C₆ alkyl. In another embodiment, the alkyl of the 4-(S-alkyl)cyclophosphamide is ethyl. The synthesis of 4-(S-ethyl)cyclophosphamide is described, for example, in G. Peter, et al., Cancer Treatment Reports, 60(4): 429-435 (1976), supra.

Cyclophosphamide or a cyclophosphamide derivative can be gonadotoxic, thus potentially reducing a subject's fertility or putting a female subject, who subsequently becomes pregnant, at high risk for spontaneous abortion, preterm labor, and/or delivery of low birth weight infants. Therefore, in some embodiments the subject is not pregnant or at risk of pregnancy. If the female subject is at risk for pregnancy, the methods can further comprise administration of a gonadotropin releasing hormone agonistic analog (“GnRH-a”), as described, for example, in Z. Blumenfeld, “Gender difference: fertility preservation in young women but not in men exposed to gonadotoxic chemotherapy.” Minerva Endocrinol., 32: 23-34 (2007), the contents of which are incorporated by reference herein in their entirety. In one embodiment, the GnRH-a is goserelin acetate, leuprolide acetate, or nafarelin acetate. In one embodiment, administration of the GnRH-a reduces any gonadotoxic effect of the cyclophosphamide or cyclophosphamide derivative. In some embodiments, the GnRH-a is administered concurrently with the cyclophosphamide or cyclophosphamide derivative. In other embodiments, the GnRH-a is administered monthly starting about 4 to 6 months before the first dose of cyclophosphamide or cyclophosphamide derivative, and continuing until the final administration of the cyclophosphamide or cyclophosphamide derivative. In some embodiments, the GnRH-a is administered monthly at an amount of about 3 to about 4 mg.

In some embodiments, the methods further comprise administering an effective amount of an anti-lymphocyte antibody to the subject. In one embodiment, the anti-lymphocyte antibody is administered concurrently with or after the administration of the cyclophosphamide or cyclophosphamide derivative. In another embodiment, the anti-lymphocyte antibody reduces the number of the subject's T cells. Suitable anti-lymphocyte antibodies include, but are not limited to, anti-thymocyte globulin (“ATG”), anti-CD3 lymphocyte antigen, anti-CD4 lymphocyte antigen, anti-CD8 lymphocyte antigen, anti-lymphocyte serum, anti-natural killer (“NK”) cell antibodies, and anti-CD40L antibodies. In some embodiments, the anti-lymphocyte antibody is ATG. In other embodiments, the anti-lymphocyte antibody is not ATG.

In some embodiments, the anti-lymphocyte antibody is administered at an amount of about 1 to about 20 mg/kg/day. In other embodiments, the anti-lymphocyte antibody is administered at an amount of about 10 to about 20 mg/kg/day. In other embodiments, the anti-lymphocyte antibody is administered at an amount of about 1.5 to about 2.5 mg/kg/day. In other embodiments, the anti-lymphocyte antibody is administered at an amount of about 2.5 mg/kg/day. In other embodiments, the anti-lymphocyte antibody is administered at an amount of about 1.5 mg/kg/day. In some embodiments, the anti-lymphocyte antibody is administered daily for a period of about 1 to about 2, 3, 4, 5, 6, 7, 8, 9, 10, 11, 12, 13, and/or about 14 days. In other embodiments, the anti-lymphocyte antibody is administered daily for a period of about 3 to about 4, 5, and/or about 6 days. In other embodiments, the anti-lymphocyte antibody is administered daily for a period of about 4 days. In still other embodiments, the anti-lymphocyte antibody is administered daily for a period of about 4 days at an amount of about 2.5 mg/kg/day. In some embodiments, the anti-lymphocyte antibody is administered intravenously.

In some embodiments, the anti-lymphocyte antibody is administered concurrently with the administration of the cyclophosphamide or cyclophosphamide derivative. In some embodiments, each dose of anti-lymphocyte antibody is administered on the same day that a dose of cyclophosphamide or cyclophosphamide derivative is administered. In other embodiments, the anti-lymphocyte antibody is administered after the administration of the first or second dose of cyclophosphamide or cyclophosphamide derivative.

In other embodiments, the anti-lymphocyte antibody is administered after the administration of the cyclophosphamide or cyclophosphamide derivative. In some embodiments, the first dose of anti-lymphocyte antibody is administered about 0 to about 1, 2, 3, 4, 5, and/or about 6 days after the administration of the final dose of cyclophosphamide or cyclophosphamide derivative. In other embodiments, the anti-lymphocyte antibody is administered about 6 days after the administration of the final dose of cyclophosphamide or cyclophosphamide derivative. In other embodiments, the anti-lymphocyte antibody is administered subsequent to when the cyclophosphamide or cyclophosphamide derivative achieves immune lymphablation.

In other embodiments, the methods further comprise administering an effective amount of granulocyte colony stimulating factor (“GCSF”) to the subject. In one embodiment, the GCSF is administered at an amount that is effective to promote reconstitution of the immune system. Reconstitution of the immune system is achieved when the subject's absolute neutrophil count exceeds 1.0×10⁹ cells/L of blood. Techniques for determining absolute neutrophil count are well known to persons skilled in the art.

The GCSF can be administered prior to, subsequent to, or concurrently with the cyclophosphamide or cyclophosphamide derivative. In one embodiment, the GCSF is administered subsequent to when the cyclophosphamide or cyclophosphamide derivative achieves immune lymphablation. In some embodiments, the GCSF is administered to the subject about 2 to about 3, 4, 5, 6, 7, and/or about 8 days after the administration of a dose of cyclophosphamide or cyclophosphamide derivative. In another embodiment, administration of cyclophosphamide or cyclophosphamide derivative is discontinued prior to administering GCSF. In some embodiments, the GCSF is administered to the subject about 6 days after the administration of the final dose of cyclophosphamide or cyclophosphamide derivative. In one embodiment, the GCSF is administered intravenously or subcutaneously.

In some embodiments, the GCSF is administered at an amount of about 2 to about 10 μkg/day. In other embodiments, the GCSF is administered at an amount of about 5 μg/kg/day. In some embodiments, the GCSF is administered to the subject until the subject's absolute neutrophil count exceeds 1.0×10⁹ cells/L of blood. In other embodiments, the GCSF is administered daily for a period of about 2 days.

In other embodiments, the methods further comprise administering an effective amount of one or more antibiotics to the subject. In one embodiment, the antibiotic is administered at an amount that is effective to minimize or prevent infection during reconstitution of the immune system. The antibiotic can be administered before, during, or after the administration of the cyclophosphamide or cyclophosphamide derivative. Suitable antibiotics include, but are not limited to, norfloxacin, fluconaxole, valacyclovir, ciprofloxacin, metronidazole, nitroimidazoles, clofazimine, clarithromycin, rifabutin, rifaximin, rifampicin, sulfadoxine, ethambutol and levofloxacin.

### Preventing Re-Activation of the Autoimmune Disease

The immunomodulatory agents of the invention can be administered at an amount effective to reduce the likelihood or prevent re-activation of the autoimmune disease. Re-activation of the autoimmune disease, as used herein, means the appearance of one or more clinical or pathological indicators of the autoimmune disease. Clinical or pathological indicators of Crohn's disease, for example, include diarrhea, gastrointestinal, bleeding, and/or abdominal pain. Clinical or pathological indicators of MS, for example, include changes in sensation (e.g. hypoesthesias and paraesthesias), muscle weakness, muscle spasms, difficulty moving; difficulty with coordination and/or balance (e.g. ataxia); difficulty speaking (e.g. dysarthria), difficulty swallowing (e.g. dysphagia), difficulty controlling eye movement (e.g. nystagmus), impaired vision (e.g. diplopia), fatigue, pain (e.g. acute or chronic), difficulty controlling bladder function, difficulty controlling bowel function, and/or depression. In some embodiments, the autoimmune disease is in remission in the subject. In certain embodiments, the remission is a result of immune lymphablation. In some embodiments, the subject has never had the autoimmune disease.

In some embodiments, the immunomodulatory agent of the invention can be an amino acid co-polymer or a pharmaceutically acceptable salt thereof, a peptide fragment of myelin basic protein or a pharmaceutically acceptable salt thereof, or an anti-tumor necrosis factor agent. When the immunomodulatory agent is administered with cyclophosphamide or a cyclophosphamide derivative, it can be administered to the subject before, during, or after the administration of cyclophosphamide or the cyclophosphamide derivative.

In one embodiment, the additional immunomodulatory agent is an amino acid co-polymer or a pharmaceutically acceptable salt thereof. Suitable amino acid co-polymers include, but are not limited to, amino acid co-polymers of: (i) tyrosine, glutamic acid, alanine, and lysine (“YEAK”); (ii) valine, tryptophan, alanine, and lysine (“VWAK”); (iii) phenylalanine, tyrosine, alanine, and lysine (“FYAK”); (iv) tyrosine, phenylalanine, alanine, and lysine (“YFAK”); and (v) valine, tyrosine, alanine, and lysine (“VYAK”). Suitable amino acid co-polymers also include those described in U.S. Pat. No. 7,381,790, the contents of which are incorporated by reference herein in their entirety. In one embodiment, the YEAK amino acid co-polymer is glatiramer. In one embodiment, the amino acid co-polymer is in the form of a pharmaceutically acceptable salt. In another embodiment, the pharmaceutically acceptable salt of the glatiramer is glatiramer acetate, also known as copolymer-1 (see U.S. Pat. Nos. 5,981,589; 6,054,430; 6,342,476; 6,362,161; 6,620,847; 6,939,539; and 7,199,098, the contents of each of which are incorporated by reference herein in their entirety). In one embodiment, the glatiramer acetate is in the form of a composition. In one embodiment, the composition is that which is sold under the trademark Copaxone®. In another embodiment, the amino acid co-polymer is not glatiramer or glatiramer acetate.

In some embodiments, the amino acid co-polymer or pharmaceutically acceptable salt thereof is administered in an amount of about 20 to about 40 mg/kg/day. In other embodiments, the amino acid co-polymer or pharmaceutically acceptable salt thereof is administered in an amount of about 20 mg/kg/day. In some embodiments, the amino acid co-polymer or pharmaceutically acceptable salt thereof is administered daily for a period of at least about 30 days. In some embodiments, the amino acid co-polymer or pharmaceutically acceptable salt thereof is administered daily for a period of about 30 days to about 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360 and/or to about 365 days.

In some embodiments, the amino acid co-polymer or pharmaceutically acceptable salt thereof is administered before, concurrently with, or after the administration of the cyclophosphamide or cyclophosphamide derivative. In some embodiments, the first dose of amino acid co-polymer or pharmaceutically acceptable salt thereof is administered about 0 to about 1, 2, 3, 4, 5, 6, 7, 8, 9, 10, 11, 12, 13, 14, 15, 16, 17, 18, 19, 20, 21, 22, 23, 24, 25, 26, 27, 28, 29, and/or 30 days after the administration of the last dose of cyclophosphamide or cyclophosphamide derivative. In some embodiments, the first dose of amino acid co-polymer or pharmaceutically acceptable salt thereof is administered about 30 days after the administration of the last dose of cyclophosphamide or cyclophosphamide derivative. In other embodiments, the first dose of amino acid co-polymer or pharmaceutically acceptable salt thereof is administered about 0 to about 30 days, e.g., about 1, 2, 3, 4, 5, 6, 7, 8, 9, 10, 11, 12, 13, 14, 15, 16, 17, 18, 19, 20, 21, 22; 23, 24, 25, 26, 27, 28, 29, and/or 30 days, before the administration of the first dose of cyclophosphamide or cyclophosphamide derivative. In some embodiments, the amino acid co-polymer or pharmaceutically acceptable salt thereof is administered about 30 days to about 1 year, e.g., about 1 month, 2 months, about 3 months, about 4 months, about 5 months, about 6 months, about 7 months, about 8 months, about 9 months, about 10 months, about 11 months to about 12 months or more. In some embodiments, the amino acid co-polymer or pharmaceutically acceptable salt thereof is administered subcutaneously.

In one embodiment, the additional immunomodulatory agent is a peptide fragment of myelin basic protein or a pharmaceutically acceptable salt thereof. As used herein, a “peptide fragment of myelin basic protein” means a peptide having an amino acid sequence that corresponds to a contiguous span of at least 10 amino acids of myelin basic protein. Myelin basic protein has the following sequence: Ala-Ser-Gln-Lys-Arg-Pro-Ser-Gln-Arg-His-Gly-Ser-Lys-Tyr-Leu-Ala-Thr-Ala-Ser-Thr-Met-Asp-His-Ala:Arg-His-Gly-Phe-Leu-Pro-Arg-His-Arg-Asp-Thr-Gly-Ile-Leu-Asp-Ser-Ile-Gly-Arg-Phe-Phe-Gly-Gly-Asp-Arg-Gly-Ala-Pro-Lys-Arg-Gly-Ser-Gly-Lys-Asp-Ser-His-His-Pro-Ala-Arg-Thr-Ala-His-Tyr-Gly-Ser-Leu-Pro-Gln-Lys-Ser-His-Gly-Arg-Thr-Gln-Asp-Glu-Asn-Pro-Val-Val-His-Phe-Phe-Lys-Asn-Ile-Val-Thr-Pro-Arg-Thr-Pro-Pro-Pro-Ser-Gln-Gly-Lys-Gly-Arg-Gly-Leu-er-Leu-Ser-Arg-Phe-Ser-Trp-Gly-Ala-Glu-Gly-Gln-Arg-Pro-Gly-Phe-Gly-Tyr-Gly-Gly-Arg-Ala-Ser-Asp-Tyr-Lys-Ser-Ala-His-Lys-Gly-Phe-Lys-Gly-Val-Asp-Ala-Gln-Gly-Thr-Leu-Ser-Lys-Ile-Phe-Lys-Leu-Gly-Gly-Arg-Asp-Ser-Arg-Ser-Gly-Ser-Pro-Met-Ala-Arg-Arg (SEQ ID NO: 1).

Suitable peptide fragments of myelin basic protein include, but are not limited to, those listed in Table 2 below, as well as pharmaceutically acceptable salts thereof.

TABLE 2
Peptide fragments of myelin basic protein

| | |
|---|---|
| SEQ ID NO: 2 | Asp-Glu-Asn-Pro-Val-Val-His-Phe-Phe- |
| | Lys-Asn-Ile-Val-Thr-Pro-Arg-Thr |
| SEQ ID NO: 3 | Lys-Ser-His-Gly-Arg-Thr-Gln-Asp-Glu- |
| | Asn-Pro-Val-Val-His-Phe-Phe-Lys-Asn- |
| | Ile-Val-Thr |
| SEQ ID NO: 4 | Ala-Arg-Thr-Ala-His-Tyr-Gly-Ser-Leu- |
| | Pro-Gln-Lys-Ser-His-Gly |
| SEQ ID NO: 5 | His-His-Pro-Ala-Arg-Thr-Ala-His-Tyr- |
| | Gly-Ser-Leu-Pro-Gln-Lys |
| SEQ ID NO: 6 | Tyr-Gly-Ser-Leu-Pro-Gln-Lys-Ser-His- |
| | Gly-Arg-Thr-Gln-Asp-Glu |
| SEQ ID NO: 7 | Thr-Gln-Asp-Glu-Asn-Pro-Val-Val-His- |
| | Phe-Phe-Lys-Asn-Ile-Val-Thr-Pro-Arg |
| SEQ ID NO: 8 | Lys-Asn-Ile-Val-Thr-Pro-Arg-Thr-Pro- |
| | Pro-Pro-Ser-Gln-Gly-Lys-Gly |
| SEQ ID NO: 9 | Asn-Pro-Val-Val-His-Phe-Phe-Lys-Asn- |
| | Ile |
| SEQ ID NO: 10 | Pro-Val-Val-His-Phe-Phe-Lys-Asn-Ile- |
| | Val |
| SEQ ID NO: 11 | Val-Val-His-Phe-Phe-Lys-Asn-Ile-Val- |
| | Thr |
| SEQ ID NO: 12 | Val-His-Phe-Phe-Lys-Asn-Ile-Val-Thr- |
| | Pro |

Suitable peptide fragments of myelin basic protein also include those described in U.S. Pat. No. 5,817,629, the contents of which are incorporated by reference herein in its entirety. In one embodiment, the peptide fragment of myelin basic protein is a peptide of SEQ ID. NO: 2 (also known as dirucotide), or a pharmaceutically acceptable salt thereof.

In some embodiments, the peptide fragment of myelin basic protein or pharmaceutically acceptable salt thereof is administered at an amount of about 1 to about 10 mg/kg/day. In other embodiments, the peptide fragment of myelin basic protein or pharmaceutically acceptable salt thereof is administered at an amount of about 5 to about 10 mg/kg/day.

In some embodiments, the peptide fragment of myelin basic protein or pharmaceutically acceptable salt thereof is administered for a period of at least about 30 days. In some embodiments, the peptide fragment of myelin basic protein or pharmaceutically acceptable salt thereof is administered for a period of about 30 days to about 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360 and/or to about 365 days.

In some embodiments, the peptide fragment of myelin basic protein or pharmaceutically acceptable salt thereof is administered before, concurrently with, or after the administration of the cyclophosphamide or cyclophosphamide derivative. In some embodiments, the first dose of peptide fragment of myelin basic protein or pharmaceutically acceptable salt thereof is administered about 0 to about 30 days, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, and/or 30 days, after the administration of the final dose of cyclophosphamide or cyclophosphamide derivative. In some embodiments, the first dose of peptide fragment of myelin basic protein or pharmaceutically acceptable salt thereof is administered about 30 days after the administration of the final dose of cyclophosphamide or cyclophosphamide derivative. In other embodiments; the first dose of peptide fragment of myelin basic protein or pharmaceutically acceptable salt thereof is administered about 0 to about 30 days, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, and/or 30 days, before the administration of the first dose of cyclophosphamide or cyclophosphamide derivative. In some embodiments, the peptide fragment of myelin basic protein or pharmaceutically acceptable salt thereof is administered for about 30 days to about 1 year, e.g., about 1 month, about 2 months, about 3 months, about 4 months, about 5 months, about 6 months, about 7 months, about 8 months, about 9 months, about 10 months, about 11 months to about 12 months or more. In some embodiments, the peptide fragment of myelin basic protein or pharmaceutically acceptable salt thereof is administered, for example, intravenously, intrathecally, or orally.

In one embodiment, the additional immunomodulatory agent is an anti-tumor necrosis factor (“TNF”) agent. In one embodiment, the anti-TNF agent is a monoclonal antibody. Suitable anti-TNF agents include, but are not limited to, infliximab, adalimumab, certolizumab, and golimumab.

In some embodiments, the anti-TNF agent is administered at an amount of about 1 to about 20 mg/kg/day. In other embodiments, the anti-TNF agent is administered at an amount of about 5 to about 10 mg/kg/day. In other embodiments, the anti-TNF agent is administered at an amount of about 5 mg/kg/day. In some embodiments, the anti-TNF agent is administered for a period of at least about 30 days. In other embodiments, the anti-TNF agent is administered for a period of about 30 days to about 1 year. In other embodiments, the anti-TNF agent is administered every 2 to 10 weeks.

In some embodiments, the anti-TNF agent is administered after the administration of the cyclophosphamide or cyclophosphamide derivative. In some embodiments, the first dose of anti-TNF agent is administered at least about 30 days after the administration of the final dose of cyclophosphamide or cyclophosphamide derivative. In some embodiments, the first dose of anti-TNF agent is administered about 30 days after the administration of the final dose of cyclophosphamide or cyclophosphamide derivative. In other embodiments, the first dose of anti-TNF agent is administered before or upon reactivation of the autoimmune disease. In some embodiments, the anti-TNF agent is administered, for example, subcutaneously or intravenously.

In some embodiments, the methods further comprise the administration of vitamin D. Suitable forms of vitamin D include, but are not limited to vitamin D₁ (1:1 ergocalciferol:lumisterol), vitamin D₂ (ergocalciferol), vitamin D₃ (cholecalciferol), vitamin D₄ (22-dihydroergocalciferol), and vitamin D₅ (sitocalciferol). In some embodiments, the vitamin D is administered concurrently with the additional immunomodulatory agent, for example, to aid in preventing re-activation of the autoimmune disease. In other embodiments, the vitamin D is administered at an amount of about 1,000 to about 10,000 IU (international units) daily. In other embodiments, the vitamin D is administered at an amount of about 1,000 to about 4,000 IU daily.

Illustrative combinations provided by the invention are provided in Table 3 below.

TABLE 3
Illustrative combinations provided by the invention

| | | | | | | Preventing Re-activation of | |
|---|---|---|---|---|---|---|---|
| # | Achieving Immune Lymphablation | | | | | the Autoimmune Disease | |
| 1 | Cyclophosphamide | | | | | Amino acid co- | |
| | or | | | | | polymer or | |
| | cyclophosphamide | | | | | pharmaceutically | |
| | derivative | | | | | acceptable salt | |
| | | | | | | thereof | |
| 2 | Cyclophosphamide | | | | | Amino acid co- | Vitamin D |
| | or | | | | | polymer or | |
| | cyclophosphamide | | | | | pharmaceutically | |
| | derivative | | | | | acceptable salt | |
| | | | | | | thereof | |
| 3 | Cyclophosphamide | | | | | Peptide fragment | |
| | or | | | | | of myelin basic | |
| | cyclophosphamide | | | | | protein or | |
| | derivative | | | | | pharmaceutically | |
| | | | | | | acceptable salt | |
| | | | | | | thereof | |
| 4 | Cyclophosphamide | | | | | Peptide fragment | Vitamin D |
| | or | | | | | of myelin basic | |
| | cyclophosphamide | | | | | protein or | |
| | derivative | | | | | pharmaceutically | |
| | | | | | | acceptable salt | |
| | | | | | | thereof | |
| 5 | Cyclophosphamide | | | | | Anti-TNF agent | |
| | or | | | | | | |
| | cyclophosphamide | | | | | | |
| | derivative | | | | | | |
| 6 | Cyclophosphamide | | | | | Anti-TNF agent | Vitamin D |
| | or | | | | | | |
| | cyclophosphamide | | | | | | |
| | derivative | | | | | | |
| 7 | Cyclophosphamide | Anti- | | | | Amino acid co- | |
| | or | lymphocyte | | | | polymer or | |
| | cyclophosphamide | antibody | | | | pharmaceutically | |
| | derivative | | | | | acceptable salt | |
| | | | | | | thereof | |
| 8 | Cyclophosphamide | Anti- | | | | Amino acid co- | Vitamin D |
| | or | lymphocyte | | | | polymer or | |
| | cyclophosphamide | antibody | | | | pharmaceutically | |
| | derivative | | | | | acceptable salt | |
| | | | | | | thereof | |
| 9 | Cyclophosphamide | Anti- | | | | Peptide fragment | |
| | or | lymphocyte | | | | of myelin basic | |
| | cyclophosphamide | antibody | | | | protein or | |
| | derivative | | | | | pharmaceutically | |
| | | | | | | acceptable salt | |
| | | | | | | thereof | |
| 10 | Cyclophosphamide | Anti- | | | | Peptide fragment | Vitamin D |
| | or | lymphocyte | | | | of myelin basic | |
| | cyclophosphamide | antibody | | | | protein or | |
| | derivative | | | | | pharmaceutically | |
| | | | | | | acceptable salt | |
| | | | | | | thereof | |
| 11 | Cyclophosphamide | Anti- | | | | Anti-TNF agent | |
| | or | lymphocyte | | | | | |
| | cyclophosphamide | antibody | | | | | |
| | derivative | | | | | | |
| 12 | Cyclophosphamide | Anti- | | | | Anti-TNF agent | Vitamin D |
| | or | lymphocyte | | | | | |
| | cyclophosphamide | antibody | | | | | |
| | derivative | | | | | | |
| 13 | Cyclophosphamide | | GnRH-a | | | Amino acid co- | |
| | or | | | | | polymer or | |
| | cyclophosphamide | | | | | pharmaceutically | |
| | derivative | | | | | acceptable salt | |
| | | | | | | thereof | |
| 14 | Cyclophosphamide | | GnRH-a | | | Amino acid co- | Vitamin D |
| | or | | | | | polymer or | |
| | cyclophosphamide | | | | | pharmaceutically | |
| | derivative | | | | | acceptable salt | |
| | | | | | | thereof | |
| 15 | Cyclophosphamide | | GnRH-a | | | Peptide fragment | |
| | or | | | | | of myelin basic | |
| | cyclophosphamide | | | | | protein or | |
| | derivative | | | | | pharmaceutically | |
| | | | | | | acceptable salt | |
| | | | | | | thereof | |
| 16 | Cyclophosphamide | | GnRH-a | | | Peptide fragment | Vitamin D |
| | or | | | | | of myelin basic | |
| | cyclophosphamide | | | | | protein or | |
| | derivative | | | | | pharmaceutically | |
| | | | | | | acceptable salt | |
| | | | | | | thereof | |
| 17 | Cyclophosphamide | | GnRH-a | | | Anti-TNF agent | |
| | or | | | | | | |
| | cyclophosphamide | | | | | | |
| | derivative | | | | | | |
| 18 | Cyclophosphamide | | GnRH-a | | | Anti-TNF agent | Vitamin D |
| | or | | | | | | |
| | cyclophosphamide | | | | | | |
| | derivative | | | | | | |
| 19 | Cyclophosphamide | | | GCSF | | Amino acid co- | |
| | or | | | | | polymer or | |
| | cyclophosphamide | | | | | pharmaceutically | |
| | derivative | | | | | acceptable salt | |
| | | | | | | thereof | |
| 20 | Cyclophosphamide | | | GCSF | | Amino acid co- | Vitamin D |
| | or | | | | | polymer or | |
| | cyclophosphamide | | | | | pharmaceutically | |
| | derivative | | | | | acceptable salt | |
| | | | | | | thereof | |
| 21 | Cyclophosphamide | | | GCSF | | Peptide fragment | |
| | or | | | | | of myelin basic | |
| | cyclophosphamide | | | | | protein or | |
| | derivative | | | | | pharmaceutically | |
| | | | | | | acceptable salt | |
| | | | | | | thereof | |
| 22 | Cyclophosphamide | | | GCSF | | Peptide fragment | Vitamin D |
| | or | | | | | of myelin basic | |
| | cyclophosphamide | | | | | protein or | |
| | derivative | | | | | pharmaceutically | |
| | | | | | | acceptable salt | |
| | | | | | | thereof | |
| 23 | Cyclophosphamide | | | GCSF | | Anti-TNF agent | |
| | or | | | | | | |
| | cyclophosphamide | | | | | | |
| | derivative | | | | | | |
| 24 | Cyclophosphamide | | | GCSF | | Anti-TNF agent | Vitamin D |
| | or | | | | | | |
| | cyclophosphamide | | | | | | |
| | derivative | | | | | | |
| 25 | Cyclophosphamide | | | | Antibiotic | Amino acid co- | |
| | or | | | | | polymer or | |
| | cyclophosphamide | | | | | pharmaceutically | |
| | derivative | | | | | acceptable salt | |
| | | | | | | thereof | |
| 26 | Cyclophosphamide | | | | Antibiotic | Amino acid co- | Vitamin D |
| | or | | | | | polymer or | |
| | cyclophosphamide | | | | | pharmaceutically | |
| | derivative | | | | | acceptable salt | |
| | | | | | | thereof | |
| 27 | Cyclophosphamide | | | | Antibiotic | Peptide fragment | |
| | or | | | | | of myelin basic | |
| | cyclophosphamide | | | | | protein or | |
| | derivative | | | | | pharmaceutically | |
| | | | | | | acceptable salt | |
| | | | | | | thereof | |
| 28 | Cyclophosphamide | | | | Antibiotic | Peptide fragment | Vitamin D |
| | or | | | | | of myelin basic | |
| | cyclophosphamide | | | | | protein or | |
| | derivative | | | | | pharmaceutically | |
| | | | | | | acceptable salt | |
| | | | | | | thereof | |
| 29 | Cyclophosphamide | | | | Antibiotic | Anti-TNF agent | |
| | or | | | | | | |
| | cyclophosphamide | | | | | | |
| | derivative | | | | | | |
| 30 | Cyclophosphamide | | | | Antibiotic | Anti-TNF agent | Vitamin D |
| | or | | | | | | |
| | cyclophosphamide | | | | | | |
| | derivative | | | | | | |
| 31 | Cyclophosphamide | Anti- | GnRH-a | | | Amino acid co- | |
| | or | lymphocyte | | | | polymer or | |
| | cyclophosphamide | antibody | | | | pharmaceutically | |
| | derivative | | | | | acceptable salt | |
| | | | | | | thereof | |
| 32 | Cyclophosphamide | Anti- | GnRH-a | | | Amino acid co- | Vitamin D |
| | or | lymphocyte | | | | polymer or | |
| | cyclophosphamide | antibody | | | | pharmaceutically | |
| | derivative | | | | | acceptable salt | |
| | | | | | | thereof | |
| 33 | Cyclophosphamide | Anti- | GnRH-a | | | Peptide fragment | |
| | or | lymphocyte | | | | of myelin basic | |
| | cyclophosphamide | antibody | | | | protein or | |
| | derivative | | | | | pharmaceutically | |
| | | | | | | acceptable salt | |
| | | | | | | thereof | |
| 34 | Cyclophosphamide | Anti- | GnRH-a | | | Peptide fragment | Vitamin D |
| | or | lymphocyte | | | | of myelin basic | |
| | cyclophosphamide | antibody | | | | protein or | |
| | derivative | | | | | pharmaceutically | |
| | | | | | | acceptable salt | |
| | | | | | | thereof | |
| 35 | Cyclophosphamide | Anti- | GnRH-a | | | Anti-TNF agent | |
| | or | lymphocyte | | | | | |
| | cyclophosphamide | antibody | | | | | |
| | derivative | | | | | | |
| 36 | Cyclophosphamide | Anti- | GnRH-a | | | Anti-TNF agent | Vitamin D |
| | or | lymphocyte | | | | | |
| | cyclophosphamide | antibody | | | | | |
| | derivative | | | | | | |
| 37 | Cyclophosphamide | Anti- | | GCSF | | Amino acid co- | |
| | or | lymphocyte | | | | polymer or | |
| | cyclophosphamide | antibody | | | | pharmaceutically | |
| | derivative | | | | | acceptable salt | |
| | | | | | | thereof | |
| 38 | Cyclophosphamide | Anti- | | GCSF | | Amino acid co- | Vitamin D |
| | or | lymphocyte | | | | polymer or | |
| | cyclophosphamide | antibody | | | | pharmaceutically | |
| | derivative | | | | | acceptable salt | |
| | | | | | | thereof | |
| 39 | Cyclophosphamide | Anti- | | GCSF | | Peptide fragment | |
| | or | lymphocyte | | | | of myelin basic | |
| | cyclophosphamide | antibody | | | | protein or | |
| | derivative | | | | | pharmaceutically | |
| | | | | | | acceptable salt | |
| | | | | | | thereof | |
| 40 | Cyclophosphamide | Anti- | | GCSF | | Peptide fragment | Vitamin D |
| | or | lymphocyte | | | | of myelin basic | |
| | cyclophosphamide | antibody | | | | protein or | |
| | derivative | | | | | pharmaceutically | |
| | | | | | | acceptable salt | |
| | | | | | | thereof | |
| 41 | Cyclophosphamide | Anti- | | GCSF | | Anti-TNF agent | |
| | or | lymphocyte | | | | | |
| | cyclophosphamide | antibody | | | | | |
| | derivative | | | | | | |
| 42 | Cyclophosphamide | Anti- | | GCSF | | Anti-TNF agent | Vitamin D |
| | or | lymphocyte | | | | | |
| | cyclophosphamide | antibody | | | | | |
| | derivative | | | | | | |
| 43 | Cyclophosphamide | Anti- | | | Antibiotic | Amino acid co- | |
| | or | lymphocyte | | | | polymer or | |
| | cyclophosphamide | antibody | | | | pharmaceutically | |
| | derivative | | | | | acceptable salt | |
| | | | | | | thereof | |
| 44 | Cyclophosphamide | Anti- | | | Antibiotic | Amino acid co- | Vitamin D |
| | or | lymphocyte | | | | polymer or | |
| | cyclophosphamide | antibody | | | | pharmaceutically | |
| | derivative | | | | | acceptable salt | |
| | | | | | | thereof | |
| 45 | Cyclophosphamide | Anti- | | | Antibiotic | Peptide fragment | |
| | or | lymphocyte | | | | of myelin basic | |
| | cyclophosphamide | antibody | | | | protein or | |
| | derivative | | | | | pharmaceutically | |
| | | | | | | acceptable salt | |
| | | | | | | thereof | |
| 46 | Cyclophosphamide | Anti- | | | Antibiotic | Peptide fragment | Vitamin D |
| | or | lymphocyte | | | | of myelin basic | |
| | cyclophosphamide | antibody | | | | protein or | |
| | derivative | | | | | pharmaceutically | |
| | | | | | | acceptable salt | |
| | | | | | | thereof | |
| 47 | Cyclophosphamide | Anti- | | | Antibiotic | Anti-TNF agent | |
| | or | lymphocyte | | | | | |
| | cyclophosphamide | antibody | | | | | |
| | derivative | | | | | | |
| 48 | Cyclophosphamide | Anti- | | | Antibiotic | Anti-TNF agent | Vitamin D |
| | or | lymphocyte | | | | | |
| | cyclophosphamide | antibody | | | | | |
| | derivative | | | | | | |
| 49 | Cyclophosphamide | | GnRH-a | GCSF | | Amino acid co- | |
| | or | | | | | polymer or | |
| | cyclophosphamide | | | | | pharmaceutically | |
| | derivative | | | | | acceptable salt | |
| | | | | | | thereof | |
| 50 | Cyclophosphamide | | GnRH-a | GCSF | | Amino acid co- | Vitamin D |
| | or | | | | | polymer or | |
| | cyclophosphamide | | | | | pharmaceutically | |
| | derivative | | | | | acceptable salt | |
| | | | | | | thereof | |
| 51 | Cyclophosphamide | | GnRH-a | GCSF | | Peptide fragment | |
| | or | | | | | of myelin basic | |
| | cyclophosphamide | | | | | protein or | |
| | derivative | | | | | pharmaceutically | |
| | | | | | | acceptable salt | |
| | | | | | | thereof | |
| 52 | Cyclophosphamide | | GnRH-a | GCSF | | Peptide fragment | Vitamin D |
| | or | | | | | of myelin basic | |
| | cyclophosphamide | | | | | protein or | |
| | derivative | | | | | pharmaceutically | |
| | | | | | | acceptable salt | |
| | | | | | | thereof | |
| 53 | Cyclophosphamide | | GnRH-a | GCSF | | Anti-TNF agent | |
| | or | | | | | | |
| | cyclophosphamide | | | | | | |
| | derivative | | | | | | |
| 54 | Cyclophosphamide | | GnRH-a | GCSF | | Anti-TNF agent | Vitamin D |
| | or | | | | | | |
| | cyclophosphamide | | | | | | |
| | derivative | | | | | | |
| 55 | Cyclophosphamide | | GnRH-a | | Antibiotic | Amino acid co- | |
| | or | | | | | polymer or | |
| | cyclophosphamide | | | | | pharmaceutically | |
| | derivative | | | | | acceptable salt | |
| | | | | | | thereof | |
| 56 | Cyclophosphamide | | GnRH-a | | Antibiotic | Amino acid co- | Vitamin D |
| | or | | | | | polymer or | |
| | cyclophosphamide | | | | | pharmaceutically | |
| | derivative | | | | | acceptable salt | |
| | | | | | | thereof | |
| 57 | Cyclophosphamide | | GnRH-a | | Antibiotic | Peptide fragment | |
| | or | | | | | of myelin basic | |
| | cyclophosphamide | | | | | protein or | |
| | derivative | | | | | pharmaceutically | |
| | | | | | | acceptable salt | |
| | | | | | | thereof | |
| 58 | Cyclophosphamide | | GnRH-a | | Antibiotic | Peptide fragment | Vitamin D |
| | or | | | | | of myelin basic | |
| | cyclophosphamide | | | | | protein or | |
| | derivative | | | | | pharmaceutically | |
| | | | | | | acceptable salt | |
| | | | | | | thereof | |
| 59 | Cyclophosphamide | | GnRH-a | | Antibiotic | Anti-TNF agent | |
| | or | | | | | | |
| | cyclophosphamide | | | | | | |
| | derivative | | | | | | |
| 60 | Cyclophosphamide | | GnRH-a | | Antibiotic | Anti-TNF agent | Vitamin D |
| | or | | | | | | |
| | cyclophosphamide | | | | | | |
| | derivative | | | | | | |
| 61 | Cyclophosphamide | | | GCSF | Antibiotic | Amino acid co- | |
| | or | | | | | polymer or | |
| | cyclophosphamide | | | | | pharmaceutically | |
| | derivative | | | | | acceptable salt | |
| | | | | | | thereof | |
| 62 | Cyclophosphamide | | | GCSF | Antibiotic | Amino acid co- | Vitamin D |
| | or | | | | | polymer or | |
| | cyclophosphamide | | | | | pharmaceutically | |
| | derivative | | | | | acceptable salt | |
| | | | | | | thereof | |
| 63 | Cyclophosphamide | | | GCSF | Antibiotic | Peptide fragment | |
| | or | | | | | of myelin basic | |
| | cyclophosphamide | | | | | protein or | |
| | derivative | | | | | pharmaceutically | |
| | | | | | | acceptable salt | |
| | | | | | | thereof | |
| 64 | Cyclophosphamide | | | GCSF | Antibiotic | Peptide fragment | Vitamin D |
| | or | | | | | of myelin basic | |
| | cyclophosphamide | | | | | protein or | |
| | derivative | | | | | pharmaceutically | |
| | | | | | | acceptable salt | |
| | | | | | | thereof | |
| 65 | Cyclophosphamide | | | GCSF | Antibiotic | Anti-TNF agent | |
| | or | | | | | | |
| | cyclophosphamide | | | | | | |
| | derivative | | | | | | |
| 66 | Cyclophosphamide | | | GCSF | Antibiotic | Anti-TNF agent | Vitamin D |
| | or | | | | | | |
| | cyclophosphamide | | | | | | |
| | derivative | | | | | | |
| 67 | Cyclophosphamide | Anti- | GnRH-a | GCSF | | Amino acid co- | |
| | or | lymphocyte | | | | polymer or | |
| | cyclophosphamide | antibody | | | | pharmaceutically | |
| | derivative | | | | | acceptable salt | |
| | | | | | | thereof | |
| 68 | Cyclophosphamide | Anti- | GnRH-a | GCSF | | Amino acid co- | Vitamin D |
| | or | lymphocyte | | | | polymer or | |
| | cyclophosphamide | antibody | | | | pharmaceutically | |
| | derivative | | | | | acceptable salt | |
| | | | | | | thereof | |
| 69 | Cyclophosphamide | Anti- | GnRH-a | GCSF | | Peptide fragment | |
| | or | lymphocyte | | | | of myelin basic | |
| | cyclophosphamide | antibody | | | | protein or | |
| | derivative | | | | | pharmaceutically | |
| | | | | | | acceptable salt | |
| | | | | | | thereof | |
| 70 | Cyclophosphamide | Anti- | GnRH-a | GCSF | | Peptide fragment | Vitamin D |
| | or | lymphocyte | | | | of myelin basic | |
| | cyclophosphamide | antibody | | | | protein or | |
| | derivative | | | | | pharmaceutically | |
| | | | | | | acceptable salt | |
| | | | | | | thereof | |
| 71 | Cyclophosphamide | Anti- | GnRH-a | GCSF | | Anti-TNF agent | |
| | or | lymphocyte | | | | | |
| | cyclophosphamide | antibody | | | | | |
| | derivative | | | | | | |
| 72 | Cyclophosphamide | Anti- | GnRH-a | GCSF | | Anti-TNF agent | Vitamin D |
| | or | lymphocyte | | | | | |
| | cyclophosphamide | antibody | | | | | |
| | derivative | | | | | | |
| 73 | Cyclophosphamide | Anti- | GnRH-a | | Antibiotic | Amino acid co- | |
| | or | lymphocyte | | | | polymer or | |
| | cyclophosphamide | antibody | | | | pharmaceutically | |
| | derivative | | | | | acceptable salt | |
| | | | | | | thereof | |
| 74 | Cyclophosphamide | Anti- | GnRH-a | | Antibiotic | Amino acid co- | Vitamin D |
| | or | lymphocyte | | | | polymer or | |
| | cyclophosphamide | antibody | | | | pharmaceutically | |
| | derivative | | | | | acceptable salt | |
| | | | | | | thereof | |
| 75 | Cyclophosphamide | Anti- | GnRH-a | | Antibiotic | Peptide fragment | |
| | or | lymphocyte | | | | of myelin basic | |
| | cyclophosphamide | antibody | | | | protein or | |
| | derivative | | | | | pharmaceutically | |
| | | | | | | acceptable salt | |
| | | | | | | thereof | |
| 76 | Cyclophosphamide | Anti- | GnRH-a | | Antibiotic | Peptide fragment | Vitamin D |
| | or | lymphocyte | | | | of myelin basic | |
| | cyclophosphamide | antibody | | | | protein or | |
| | derivative | | | | | pharmaceutically | |
| | | | | | | acceptable salt | |
| | | | | | | thereof | |
| 77 | Cyclophosphamide | Anti- | GnRH-a | | Antibiotic | Anti-TNF agent | |
| | or | lymphocyte | | | | | |
| | cyclophosphamide | antibody | | | | | |
| | derivative | | | | | | |
| 78 | Cyclophosphamide | Anti- | GnRH-a | | Antibiotic | Anti-TNF agent | Vitamin D |
| | or | lymphocyte | | | | | |
| | cyclophosphamide | antibody | | | | | |
| | derivative | | | | | | |
| 79 | Cyclophosphamide | Anti- | GnRH-a | GCSF | Antibiotic | Amino acid co- | |
| | or | lymphocyte | | | | polymer or | |
| | cyclophosphamide | antibody | | | | pharmaceutically | |
| | derivative | | | | | acceptable salt | |
| | | | | | | thereof | |
| 80 | Cyclophosphamide | Anti- | GnRH-a | GCSF | Antibiotic | Amino acid co- | Vitamin D |
| | or | lymphocyte | | | | polymer or | |
| | cyclophosphamide | antibody | | | | pharmaceutically | |
| | derivative | | | | | acceptable salt | |
| | | | | | | thereof | |
| 81 | Cyclophosphamide | Anti- | GnRH-a | GCSF | Antibiotic | Peptide fragment | |
| | or | lymphocyte | | | | of myelin basic | |
| | cyclophosphamide | antibody | | | | protein or | |
| | derivative | | | | | pharmaceutically | |
| | | | | | | acceptable salt | |
| | | | | | | thereof | |
| 82 | Cyclophosphamide | Anti- | GnRH-a | GCSF | Antibiotic | Peptide fragment | Vitamin D |
| | or | lymphocyte | | | | of myelin basic | |
| | cyclophosphamide | antibody | | | | protein or | |
| | derivative | | | | | pharmaceutically | |
| | | | | | | acceptable salt | |
| | | | | | | thereof | |
| 83 | Cyclophosphamide | Anti- | GnRH-a | GCSF | Antibiotic | Anti-TNF agent | |
| | or | lymphocyte | | | | | |
| | cyclophosphamide | antibody | | | | | |
| | derivative | | | | | | |
| 84 | Cyclophosphamide | Anti- | GnRH-a | GCSF | Antibiotic | Anti-TNF agent | Vitamin D |
| | or | lymphocyte | | | | | |
| | cyclophosphamide | antibody | | | | | |
| | derivative | | | | | | |
| 85 | 4-hydroperoxy | | | | | Amino acid co- | |
| | cyclophosphamide | | | | | polymer or | |
| | | | | | | pharmaceutically | |
| | | | | | | acceptable salt | |
| | | | | | | thereof | |
| 86 | 4-hydroperoxy | | | | | Amino acid co- | Vitamin D |
| | cyclophosphamide | | | | | polymer or | |
| | | | | | | pharmaceutically | |
| | | | | | | acceptable salt | |
| | | | | | | thereof | |
| 87 | 4-hydroperoxy | | | | | Peptide fragment | |
| | cyclophosphamide | | | | | of myelin basic | |
| | | | | | | protein or | |
| | | | | | | pharmaceutically | |
| | | | | | | acceptable salt | |
| | | | | | | thereof | |
| 88 | 4-hydroperoxy | | | | | Peptide fragment | Vitamin D |
| | cyclophosphamide | | | | | of myelin basic | |
| | | | | | | protein or | |
| | | | | | | pharmaceutically | |
| | | | | | | acceptable salt | |
| | | | | | | thereof | |
| 89 | 4-hydroperoxy | | | | | Anti-TNF agent | |
| | cyclophosphamide | | | | | | |
| 90 | 4-hydroperoxy | | | | | Anti-TNF agent | Vitamin D |
| | cyclophosphamide | | | | | | |
| 91 | 4-hydroperoxy | Anti- | | | | Amino acid co- | |
| | cyclophosphamide | lymphocyte | | | | polymer or | |
| | | antibody | | | | pharmaceutically | |
| | | | | | | acceptable salt | |
| | | | | | | thereof | |
| 92 | 4-hydroperoxy | Anti- | | | | Amino acid co- | Vitamin D |
| | cyclophosphamide | lymphocyte | | | | polymer or | |
| | | antibody | | | | pharmaceutically | |
| | | | | | | acceptable salt | |
| | | | | | | thereof | |
| 93 | 4-hydroperoxy | Anti- | | | | Peptide fragment | |
| | cyclophosphamide | lymphocyte | | | | of myelin basic | |
| | | antibody | | | | protein or | |
| | | | | | | pharmaceutically | |
| | | | | | | acceptable salt | |
| | | | | | | thereof | |
| 94 | 4-hydroperoxy | Anti- | | | | Peptide fragment | Vitamin D |
| | cyclophosphamide | lymphocyte | | | | of myelin basic | |
| | | antibody | | | | protein or | |
| | | | | | | pharmaceutically | |
| | | | | | | acceptable salt | |
| | | | | | | thereof | |
| 95 | 4-hydroperoxy | Anti- | | | | Anti-TNF agent | |
| | cyclophosphamide | lymphocyte | | | | | |
| | | antibody | | | | | |
| 96 | 4-hydroperoxy | Anti- | | | | Anti-TNF agent | Vitamin D |
| | cyclophosphamide | lymphocyte | | | | | |
| | | antibody | | | | | |
| 97 | 4-hydroperoxy | | GnRH-a | | | Amino acid co- | |
| | cyclophosphamide | | | | | polymer or | |
| | | | | | | pharmaceutically | |
| | | | | | | acceptable salt | |
| | | | | | | thereof | |
| 98 | 4-hydroperoxy | | GnRH-a | | | Amino acid co- | Vitamin D |
| | cyclophosphamide | | | | | polymer or | |
| | | | | | | pharmaceutically | |
| | | | | | | acceptable salt | |
| | | | | | | thereof | |
| 99 | 4-hydroperoxy | | GnRH-a | | | Peptide fragment | |
| | cyclophosphamide | | | | | of myelin basic | |
| | | | | | | protein or | |
| | | | | | | pharmaceutically | |
| | | | | | | acceptable salt | |
| | | | | | | thereof | |
| 100 | 4-hydroperoxy | | GnRH-a | | | Peptide fragment | Vitamin D |
| | cyclophosphamide | | | | | of myelin basic | |
| | | | | | | protein or | |
| | | | | | | pharmaceutically | |
| | | | | | | acceptable salt | |
| | | | | | | thereof | |
| 101 | 4-hydroperoxy | | GnRH-a | | | Anti-TNF agent | |
| | cyclophosphamide | | | | | | |
| 102 | 4-hydroperoxy | | GnRH-a | | | Anti-TNF agent | Vitamin D |
| | cyclophosphamide | | | | | | |
| 103 | 4-hydroperoxy | | | GCSF | | Amino acid co- | |
| | cyclophosphamide | | | | | polymer or | |
| | | | | | | pharmaceutically | |
| | | | | | | acceptable salt | |
| | | | | | | thereof | |
| 104 | 4-hydroperoxy | | | GCSF | | Amino acid co- | Vitamin D |
| | cyclophosphamide | | | | | polymer or | |
| | | | | | | pharmaceutically | |
| | | | | | | acceptable salt | |
| | | | | | | thereof | |
| 105 | 4-hydroperoxy | | | GCSF | | Peptide fragment | |
| | cyclophosphamide | | | | | of myelin basic | |
| | | | | | | protein or | |
| | | | | | | pharmaceutically | |
| | | | | | | acceptable salt | |
| | | | | | | thereof | |
| 106 | 4-hydroperoxy | | | GCSF | | Peptide fragment | Vitamin D |
| | cyclophosphamide | | | | | of myelin basic | |
| | | | | | | protein or | |
| | | | | | | pharmaceutically | |
| | | | | | | acceptable salt | |
| | | | | | | thereof | |
| 107 | 4-hydroperoxy | | | GCSF | | Anti-TNF agent | |
| | cyclophosphamide | | | | | | |
| 108 | 4-hydroperoxy | | | GCSF | | Anti-TNF agent | Vitamin D |
| | cyclophosphamide | | | | | | |
| 109 | 4-hydroperoxy | | | | Antibiotic | Amino acid co- | |
| | cyclophosphamide | | | | | polymer or | |
| | | | | | | pharmaceutically | |
| | | | | | | acceptable salt | |
| | | | | | | thereof | |
| 110 | 4-hydroperoxy | | | | Antibiotic | Amino acid co- | Vitamin D |
| | cyclophosphamide | | | | | polymer or | |
| | | | | | | pharmaceutically | |
| | | | | | | acceptable salt | |
| | | | | | | thereof | |
| 111 | 4-hydroperoxy | | | | Antibiotic | Peptide fragment | |
| | cyclophosphamide | | | | | of myelin basic | |
| | | | | | | protein or | |
| | | | | | | pharmaceutically | |
| | | | | | | acceptable salt | |
| | | | | | | thereof | |
| 112 | 4-hydroperoxy | | | | Antibiotic | Peptide fragment | Vitamin D |
| | cyclophosphamide | | | | | of myelin basic | |
| | | | | | | protein or | |
| | | | | | | pharmaceutically | |
| | | | | | | acceptable salt | |
| | | | | | | thereof | |
| 113 | 4-hydroperoxy | | | | Antibiotic | Anti-TNF agent | |
| | cyclophosphamide | | | | | | |
| 114 | 4-hydroperoxy | | | | Antibiotic | Anti-TNF agent | Vitamin D |
| | cyclophosphamide | | | | | | |
| 115 | 4-hydroperoxy | Anti- | GnRH-a | | | Amino acid co- | |
| | cyclophosphamide | lymphocyte | | | | polymer or | |
| | | antibody | | | | pharmaceutically | |
| | | | | | | acceptable salt | |
| | | | | | | thereof | |
| 116 | 4-hydroperoxy | Anti- | GnRH-a | | | Amino acid co- | Vitamin D |
| | cyclophosphamide | lymphocyte | | | | polymer or | |
| | | antibody | | | | pharmaceutically | |
| | | | | | | acceptable salt | |
| | | | | | | thereof | |
| 117 | 4-hydroperoxy | Anti- | GnRH-a | | | Peptide fragment | |
| | cyclophosphamide | lymphocyte | | | | of myelin basic | |
| | | antibody | | | | protein or | |
| | | | | | | pharmaceutically | |
| | | | | | | acceptable salt | |
| | | | | | | thereof | |
| 118 | 4-hydroperoxy | Anti- | GnRH-a | | | Peptide fragment | Vitamin D |
| | cyclophosphamide | lymphocyte | | | | of myelin basic | |
| | | antibody | | | | protein or | |
| | | | | | | pharmaceutically | |
| | | | | | | acceptable salt | |
| | | | | | | thereof | |
| 119 | 4-hydroperoxy | Anti- | GnRH-a | | | Anti-TNF agent | |
| | cyclophosphamide | lymphocyte | | | | | |
| | | antibody | | | | | |
| 120 | 4-hydroperoxy | Anti- | GnRH-a | | | Anti-TNF agent | Vitamin D |
| | cyclophosphamide | lymphocyte | | | | | |
| | | antibody | | | | | |
| 121 | 4-hydroperoxy | Anti- | | GCSF | | Amino acid co- | |
| | cyclophosphamide | lymphocyte | | | | polymer or | |
| | | antibody | | | | pharmaceutically | |
| | | | | | | acceptable salt | |
| | | | | | | thereof | |
| 122 | 4-hydroperoxy | Anti- | | GCSF | | Amino acid co- | Vitamin D |
| | cyclophosphamide | lymphocyte | | | | polymer or | |
| | | antibody | | | | pharmaceutically | |
| | | | | | | acceptable salt | |
| | | | | | | thereof | |
| 123 | 4-hydroperoxy | Anti- | | GCSF | | Peptide fragment | |
| | cyclophosphamide | lymphocyte | | | | of myelin basic | |
| | | antibody | | | | protein or | |
| | | | | | | pharmaceutically | |
| | | | | | | acceptable salt | |
| | | | | | | thereof | |
| 124 | 4-hydroperoxy | Anti- | | GCSF | | Peptide fragment | Vitamin D |
| | cyclophosphamide | lymphocyte | | | | of myelin basic | |
| | | antibody | | | | protein or | |
| | | | | | | pharmaceutically | |
| | | | | | | acceptable salt | |
| | | | | | | thereof | |
| 125 | 4-hydroperoxy | Anti- | | GCSF | | Anti-TNF agent | |
| | cyclophosphamide | lymphocyte | | | | | |
| | | antibody | | | | | |
| 126 | 4-hydroperoxy | Anti- | | GCSF | | Anti-TNF agent | Vitamin D |
| | cyclophosphamide | lymphocyte | | | | | |
| | | antibody | | | | | |
| 127 | 4-hydroperoxy | Anti- | | | Antibiotic | Amino acid co- | |
| | cyclophosphamide | lymphocyte | | | | polymer or | |
| | | antibody | | | | pharmaceutically | |
| | | | | | | acceptable salt | |
| | | | | | | thereof | |
| 128 | 4-hydroperoxy | Anti- | | | Antibiotic | Amino acid co- | Vitamin D |
| | cyclophosphamide | lymphocyte | | | | polymer or | |
| | | antibody | | | | pharmaceutically | |
| | | | | | | acceptable salt | |
| | | | | | | thereof | |
| 129 | 4-hydroperoxy | Anti- | | | Antibiotic | Peptide fragment | |
| | cyclophosphamide | lymphocyte | | | | of myelin basic | |
| | | antibody | | | | protein or | |
| | | | | | | pharmaceutically | |
| | | | | | | acceptable salt | |
| | | | | | | thereof | |
| 130 | 4-hydroperoxy | Anti- | | | Antibiotic | Peptide fragment | Vitamin D |
| | cyclophosphamide | lymphocyte | | | | of myelin basic | |
| | | antibody | | | | protein or | |
| | | | | | | pharmaceutically | |
| | | | | | | acceptable salt | |
| | | | | | | thereof | |
| 131 | 4-hydroperoxy | Anti- | | | Antibiotic | Anti-TNF agent | |
| | cyclophosphamide | lymphocyte | | | | | |
| | | antibody | | | | | |
| 132 | 4-hydroperoxy | Anti- | | | Antibiotic | Anti-TNF agent | Vitamin D |
| | cyclophosphamide | lymphocyte | | | | | |
| | | antibody | | | | | |
| 133 | 4-hydroperoxy | | GnRH-a | GCSF | | Amino acid co- | |
| | cyclophosphamide | | | | | polymer or | |
| | | | | | | pharmaceutically | |
| | | | | | | acceptable salt | |
| | | | | | | thereof | |
| 134 | 4-hydroperoxy | | GnRH-a | GCSF | | Amino acid co- | Vitamin D |
| | cyclophosphamide | | | | | polymer or | |
| | | | | | | pharmaceutically | |
| | | | | | | acceptable salt | |
| | | | | | | thereof | |
| 135 | 4-hydroperoxy | | GnRH-a | GCSF | | Peptide fragment | |
| | cyclophosphamide | | | | | of myelin basic | |
| | | | | | | protein or | |
| | | | | | | pharmaceutically | |
| | | | | | | acceptable salt | |
| | | | | | | thereof | |
| 136 | 4-hydroperoxy | | GnRH-a | GCSF | | Peptide fragment | Vitamin D |
| | cyclophosphamide | | | | | of myelin basic | |
| | | | | | | protein or | |
| | | | | | | pharmaceutically | |
| | | | | | | acceptable salt | |
| | | | | | | thereof | |
| 137 | 4-hydroperoxy | | GnRH-a | GCSF | | Anti-TNF agent | |
| | cyclophosphamide | | | | | | |
| 138 | 4-hydroperoxy | | GnRH-a | GCSF | | Anti-TNF agent | Vitamin D |
| | cyclophosphamide | | | | | | |
| 139 | 4-hydroperoxy | | GnRH-a | | Antibiotic | Amino acid co- | |
| | cyclophosphamide | | | | | polymer or | |
| | | | | | | pharmaceutically | |
| | | | | | | acceptable salt | |
| | | | | | | thereof | |
| 140 | 4-hydroperoxy | | GnRH-a | | Antibiotic | Amino acid co- | Vitamin D |
| | cyclophosphamide | | | | | polymer or | |
| | | | | | | pharmaceutically | |
| | | | | | | acceptable salt | |
| | | | | | | thereof | |
| 141 | 4-hydroperoxy | | GnRH-a | | Antibiotic | Peptide fragment | |
| | cyclophosphamide | | | | | of myelin basic | |
| | | | | | | protein or | |
| | | | | | | pharmaceutically | |
| | | | | | | acceptable salt | |
| | | | | | | thereof | |
| 142 | 4-hydroperoxy | | GnRH-a | | Antibiotic | Peptide fragment | Vitamin D |
| | cyclophosphamide | | | | | of myelin basic | |
| | | | | | | protein or | |
| | | | | | | pharmaceutically | |
| | | | | | | acceptable salt | |
| | | | | | | thereof | |
| 143 | 4-hydroperoxy | | GnRH-a | | Antibiotic | Anti-TNF agent | |
| | cyclophosphamide | | | | | | |
| 144 | 4-hydroperoxy | | GnRH-a | | Antibiotic | Anti-TNF agent | Vitamin D |
| | cyclophosphamide | | | | | | |
| 145 | 4-hydroperoxy | | | GCSF | Antibiotic | Amino acid co- | |
| | cyclophosphamide | | | | | polymer or | |
| | | | | | | pharmaceutically | |
| | | | | | | acceptable salt | |
| | | | | | | thereof | |
| 146 | 4-hydroperoxy | | | GCSF | Antibiotic | Amino acid co- | Vitamin D |
| | cyclophosphamide | | | | | polymer or | |
| | | | | | | pharmaceutically | |
| | | | | | | acceptable salt | |
| | | | | | | thereof | |
| 147 | 4-hydroperoxy | | | GCSF | Antibiotic | Peptide fragment | |
| | cyclophosphamide | | | | | of myelin basic | |
| | | | | | | protein or | |
| | | | | | | pharmaceutically | |
| | | | | | | acceptable salt | |
| | | | | | | thereof | |
| 148 | 4-hydroperoxy | | | GCSF | Antibiotic | Peptide fragment | Vitamin D |
| | cyclophosphamide | | | | | of myelin basic | |
| | | | | | | protein or | |
| | | | | | | pharmaceutically | |
| | | | | | | acceptable salt | |
| | | | | | | thereof | |
| 149 | 4-hydroperoxy | | | GCSF | Antibiotic | Anti-TNF agent | |
| | cyclophosphamide | | | | | | |
| 150 | 4-hydroperoxy | | | GCSF | Antibiotic | Anti-TNF agent | Vitamin D |
| | cyclophosphamide | | | | | | |
| 151 | 4-hydroperoxy | Anti- | GnRH-a | GCSF | | Amino acid co- | |
| | cyclophosphamide | lymphocyte | | | | polymer or | |
| | | antibody | | | | pharmaceutically | |
| | | | | | | acceptable salt | |
| | | | | | | thereof | |
| 152 | 4-hydroperoxy | Anti- | GnRH-a | GCSF | | Amino acid co- | Vitamin D |
| | cyclophosphamide | lymphocyte | | | | polymer or | |
| | | antibody | | | | pharmaceutically | |
| | | | | | | acceptable salt | |
| | | | | | | thereof | |
| 153 | 4-hydroperoxy | Anti- | GnRH-a | GCSF | | Peptide fragment | |
| | cyclophosphamide | lymphocyte | | | | of myelin basic | |
| | | antibody | | | | protein or | |
| | | | | | | pharmaceutically | |
| | | | | | | acceptable salt | |
| | | | | | | thereof | |
| 154 | 4-hydroperoxy | Anti- | GnRH-a | GCSF | | Peptide fragment | Vitamin D |
| | cyclophosphamide | lymphocyte | | | | of myelin basic | |
| | | antibody | | | | protein or | |
| | | | | | | pharmaceutically | |
| | | | | | | acceptable salt | |
| | | | | | | thereof | |
| 155 | 4-hydroperoxy | Anti- | GnRH-a | GCSF | | Anti-TNF agent | |
| | cyclophosphamide | lymphocyte | | | | | |
| | | antibody | | | | | |
| 156 | 4-hydroperoxy | Anti- | GnRH-a | GCSF | | Anti-TNF agent | Vitamin D |
| | cyclophosphamide | lymphocyte | | | | | |
| | | antibody | | | | | |
| 157 | 4-hydroperoxy | Anti- | GnRH-a | | Antibiotic | Amino acid co- | |
| | cyclophosphamide | lymphocyte | | | | polymer or | |
| | | antibody | | | | pharmaceutically | |
| | | | | | | acceptable salt | |
| | | | | | | thereof | |
| 158 | 4-hydroperoxy | Anti- | GnRH-a | | Antibiotic | Amino acid co- | Vitamin D |
| | cyclophosphamide | lymphocyte | | | | polymer or | |
| | | antibody | | | | pharmaceutically | |
| | | | | | | acceptable salt | |
| | | | | | | thereof | |
| 159 | 4-hydroperoxy | Anti- | GnRH-a | | Antibiotic | Peptide fragment | |
| | cyclophosphamide | lymphocyte | | | | of myelin basic | |
| | | antibody | | | | protein or | |
| | | | | | | pharmaceutically | |
| | | | | | | acceptable salt | |
| | | | | | | thereof | |
| 160 | 4-hydroperoxy | Anti- | GnRH-a | | Antibiotic | Peptide fragment | Vitamin D |
| | cyclophosphamide | lymphocyte | | | | of myelin basic | |
| | | antibody | | | | protein or | |
| | | | | | | pharmaceutically | |
| | | | | | | acceptable salt | |
| | | | | | | thereof | |
| 161 | 4-hydroperoxy | Anti- | GnRH-a | | Antibiotic | Anti-TNF agent | |
| | cyclophosphamide | lymphocyte | | | | | |
| | | antibody | | | | | |
| 162 | 4-hydroperoxy | Anti- | GnRH-a | | Antibiotic | Anti-TNF agent | Vitamin D |
| | cyclophosphamide | lymphocyte | | | | | |
| | | antibody | | | | | |
| 163 | 4-hydroperoxy | Anti- | GnRH-a | GCSF | Antibiotic | Amino acid co- | |
| | cyclophosphamide | lymphocyte | | | | polymer or | |
| | | antibody | | | | pharmaceutically | |
| | | | | | | acceptable salt | |
| | | | | | | thereof | |
| 164 | 4-hydroperoxy | Anti- | GnRH-a | GCSF | Antibiotic | Amino acid co- | Vitamin D |
| | cyclophosphamide | lymphocyte | | | | polymer or | |
| | | antibody | | | | pharmaceutically | |
| | | | | | | acceptable salt | |
| | | | | | | thereof | |
| 165 | 4-hydroperoxy | Anti- | GnRH-a | GCSF | Antibiotic | Peptide fragment | |
| | cyclophosphamide | lymphocyte | | | | of myelin basic | |
| | | antibody | | | | protein or | |
| | | | | | | pharmaceutically | |
| | | | | | | acceptable salt | |
| | | | | | | thereof | |
| 166 | 4-hydroperoxy | Anti- | GnRH-a | GCSF | Antibiotic | Peptide fragment | Vitamin D |
| | cyclophosphamide | lymphocyte | | | | of myelin basic | |
| | | antibody | | | | protein or | |
| | | | | | | pharmaceutically | |
| | | | | | | acceptable salt | |
| | | | | | | thereof | |
| 167 | 4-hydroperoxy | Anti- | GnRH-a | GCSF | Antibiotic | Anti-TNF agent | |
| | cyclophosphamide | lymphocyte | | | | | |
| | | antibody | | | | | |
| 168 | 4-hydroperoxy | Anti- | GnRH-a | GCSF | Antibiotic | Anti-TNF agent | Vitamin D |
| | cyclophosphamide | lymphocyte | | | | | |
| | | antibody | | | | | |

The cyclophosphamide or cyclophosphamide derivative or other immunomodulatory agent(s) in the above-described methods can conveniently be administered as a component of a composition that comprises a physiological carrier or vehicle.

The compositions described herein can be administered orally, by infusion, by bolus injection, by absorption through epithelial or mucocutaneous linings (e.g., oral, rectal, and intestinal mucosa), or by any other convenient route of administration. Administration can be systemic or local. Various delivery systems are known, e.g., encapsulation in liposomes, microparticles, microcapsules, capsules, and can be administered.

Methods of administration include, but are not limited to, intradermal, intramuscular, intraperitoneal, intravenous, ocular, subcutaneous, intranasal, epidural, oral, sublingual, intracerebral, intravaginal, transdermal, rectal, by inhalation, or topical, particularly to the ears, nose, eyes, or skin. In some instances, administration will result in the release of the cyclophosphamide or cyclophosphamide derivative or other immunomodulatory agent(s) into the bloodstream. The mode of administration can be left to the discretion of the practitioner.

In other embodiments, it can be desirable to administer the cyclophosphamide or cyclophosphamide derivative or other immunomodulatory agent(s) locally. This can be achieved, for example, and not by way of limitation, by local infusion during surgery, topical application, e.g., in conjunction with a wound dressing after surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the cyclophosphamide or cyclophosphamide derivative or other immunomodulatory agent(s) into the central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

Pulmonary administration can also be employed, e.g., by use of an inhaler of nebulizer, and formulation with an aerosolizing agent, or via perfusion in a fluorocarbon oar, synthetic pulmonary surfactant. In certain embodiments, the cyclophosphamide or cyclophosphamide derivative or other immunomodulatory agent(s) can be formulated as a suppository, with traditional binders and excipients such as triglycerides.

In another embodiment, the cyclophosphamide or cyclophosphamide derivative or other immunomodulatory agent(s) can be delivered in a vesicle, in particular a liposome (see Langer, Science 249:1527-1533 (1990) and Treat or prevent et al., Liposomes in the Therapy of Infectious Disease and Cancer 317-327 and 353-365 (1989), the contents of each of which are incorporated by reference herein in their entirety).

In yet another embodiment, the cyclophosphamide or cyclophosphamide derivative or other immunomodulatory agent(s) can be delivered in a controlled-release system or sustained-release system (see, e.g., Goodson, in Medical Applications of Controlled Release, supra, vol. 2, pp. 115-138 (1984), the contents of which are incorporated by reference in their entirety). Other controlled or sustained-release systems discussed in the review by Langer, Science 249:1527-1533 (1990), the contents of which are incorporated by reference herein in their entirety can be used. In one embodiment a pump can be used (Langer, Science 249:1527-1533 (1990); Sefton, CRC Crit. Ref Biomed. Eng. 14:201 (1987); Buchwald et al., Surgery 88:507 (1980); and Saudek et al., N. Engl. J. Med. 321:574 (1989), the contents of each of which are incorporated by reference herein in their entirety). In another embodiment polymeric materials can be used (see Medical Applications of Controlled Release (Langer and Wise eds., 1974), the contents of which are incorporated by reference herein in their entirety; Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989), the contents of each of which are incorporated by reference herein in their entirety).

In yet another embodiment a controlled- or sustained-release system can be placed in proximity of a target of the cyclophosphamide or cyclophosphamide derivative or other immunomodulatory agent(s), e.g., the spinal column, brain, skin, lung, thyroid gland, colon or gastrointestinal tract, thus requiring only a fraction of the systemic dose.

The compositions can optionally comprise a suitable amount of a pharmaceutically acceptable excipient so as to provide the form for proper administration to the subject. Such pharmaceutical excipients can be liquids, such as water and oils, including those of petroleum, animal, vegetable, or synthetic origin, such as peanut oil, soybean oil, mineral oil, sesame oil and the like. The pharmaceutical excipients can be saline, gum acacia, gelatin, starch paste, talc, keratin, colloidal silica, urea and the like. In addition, auxiliary, stabilizing, thickening, lubricating, and coloring agents can be used. In one embodiment the pharmaceutically acceptable excipients are sterile when administered to a subject. Water is a particularly useful excipient when the cyclophosphamide or cyclophosphamide derivative or other immunomodulatory agent(s) is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The compositions, if desired, can also contain minor amounts of wetting or emulsifying agents, or pH buffering agents.

The compositions can take the form of solutions, suspensions, emulsions, tablets, pills, pellets, capsules, capsules containing liquids, powders, sustained-release formulations, suppositories, emulsions, aerosols, sprays, suspensions, or any other form suitable for use. In one embodiment the composition is in the form of a capsule (see e.g. U.S. Pat. No. 5,698,155, incorporated herein by reference). Other examples of suitable pharmaceutical excipients are described in Remington's Pharmaceutical Sciences 1447-1676 (Alfonso R. Gennaro eds., 19th ed. 1995), incorporated herein by reference.

In one embodiment the cyclophosphamide or cyclophosphamide derivative or other immunomodulatory agent(s) are formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving a cyclophosphamide or cyclophosphamide derivative or other immunomodulatory agent(s) are also suitable for orally administered compositions. In these latter platforms, fluid from the environment surrounding the capsule is imbibed by the driving compound, which swells to displace the agent or agent composition through an aperture. These delivery platforms can provide an essentially zero-order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment the excipients are of pharmaceutical grade.

In another embodiment the cyclophosphamide or cyclophosphamide derivative or other immunomodulatory agent(s) can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized-powder or water free concentrate in a hermetically sealed container such as an ampule or sachette indicating the quantity of active agent. Where the cyclophosphamide or cyclophosphamide derivative or other immunomodulatory agent(s) are to be administered by infusion, they can be dispensed, for example, with an infusion bottle containing sterile pharmaceutical grade water or saline. Where the cyclophosphamide or cyclophosphamide derivative or other immunomodulatory agent(s) are administered by injection, an ampule of sterile water for injection or saline can be provided so that the ingredients can be mixed prior to administration.

The cyclophosphamide or cyclophosphamide derivative or other immunomodulatory agent(s) can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference. Such dosage forms can be used to provide controlled- or sustained-release of one or more active ingredients using, for example, hydropropylmethyl cellulose, other polymer matrices, gels, permeable membranes, osmotic systems, multilayer, coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

The cyclophosphamide or cyclophosphamide derivative or other immunomodulatory agent(s) can also be provided in the form of a kit to simplify the administration to the subject. A typical kit of comprises a unit dosage form of cyclophosphamide or cyclophosphamide derivative or other immunomodulatory agent(s). In some embodiments, the kit may comprise any of the compositions described herein, including the compositions listed in Table 3, and any combination thereof. In one embodiment the unit dosage form is within a container, which can be sterile, containing an effective amount of cyclophosphamide or cyclophosphamide derivative or other immunomodulatory agent(s) and a physiologically acceptable carrier or vehicle. The kit can further comprise a label or printed instructions instructing the use of the compositions of the invention, including cyclophosphamide or cyclophosphamide derivative or other immunomodulatory agent(s), to treat or prevent an autoimmune disease. The kits can further comprise a device that is useful for administering the unit dosage forms as described herein. Examples of such a device include, but are not limited to, a syringe, a drip bag, a patch, an inhaler, and an enema bag.

Having described the invention with reference to certain embodiments, other embodiments will become apparent to one skilled in the art from consideration of the specification. The invention is further defined by reference to the following examples. It will be apparent to those skilled in the art that many modifications, both to materials and methods, may be practiced without departing from the scope of the invention.

### EXAMPLES

### Example 1

### Effect of Cyclophosphamide and/or Cyclophosphamide Derivatives in Combination with Glatiramer Acetate (“GA”) on Crohn's Disease in Mice (TNBS Colitis Model)

a. Induction of Crohn's Colitis

Mice are randomized into treatment groups with the average body weight equivalent in each group. 6 to 12 week old Balb/c mice are treated with 2 mg of the hapten trinitrobenzene sulfonic acid (TNBS) in 0.1 ml of 50% ethanol by intrarectal administration using a 18 G feeding tube (Fine Science Tools, Foster City, Calif.). This treatment is repeated weekly for 5 weeks in order to induce Crohn's-like colitis.

After 5 weeks of treatment, the mice are weighed and observed for clinical signs of Crohn's-like colitis. Crohn's-like colitis is determined by weight loss, change in stool consistency, and/or microscopic blood in the stool by Hemoccult testing. Clinical signs of Crohn's-like colitis are assessed using the Daily Activity Index (“DAI”). The DAI is the average of change in weight (0, <1%; 1, 1-5%; 2, 5-10%, 23, 10-20% and 4, >20%), intestinal bleeding (0, negative; 2, microscopic blood; 4, visible blood), and stool consistency (0, normal; 2, loose stools; 4, diarrhea).

b. Administration of Cyclophosphamide and/or a Cyclophosphamide Derivative in Combination with GA

After establishment of Crohn's-like colitis (day 0), one-half of the mice are administered cyclophosphamide and/or a cyclophosphamide derivative via intraperitoneal or intravenous injection in phosphate-buffered saline (PBS) (20 mg/ml) at a dose of 100-200 mg/kg. The cyclophosphamide derivtives used in this study include 4-hydroxycyclophosphamide, aldophosphamide, 4-hydroperoxycyclophosphamide, 4-(S-ethyl)cyclophosphamide and mafosfamide. On day 2 (±2), as determined by colitis progression), GA is administered subcutaneously at a dose of 500-2000 micrograms/mouse in PBS/mannitol for up to five consecutive days.

The other half of the mice are administered with mock injections (vehicle) or intrarectal 50% ethanol as a control.

For the intravenous injections, the mice are warmed with a heat lamp (approximately 18-25 inches from the cage floor) while in their cage for 3-5 minutes to dilate their blood vessels; they are then individually restrained in a cone or Broome-type restraining device (VWR catalogue number 10718-030) for the intravenous injection administered into the lateral tail vein with a 28-30 gauge needle.

c. Results

Two months after treatment as described in step b; the mice are weighed and observed for clinical signs of Crohn's-like colitis, as described in step a above.

### Example 2

### Effect of Cyclophosphamide and/or Cyclophosphamide Derivatives in Combination with Glatiramer Acetate (“GA”) on Crohn's Disease in Mice (CD45RB^{High} Transfer Model)

a. Induction of Crohn's Colitis

CD4+ CD45RB^{high} and CD4+ CD45RB^{low} T-cell subsets are isolated from spleens of wild-type C57BL/6 female mice using immunomagnetic selection and 0.5×10⁶ cells are injected intraperitoneally to RAG −/− mice on the same background.

4 weeks after adoptive transfer, the mice are weighed and observed for clinical signs of Crohn's-like colitis. Crohn's-like colitis is determined by weight loss, change in stool consistency, and/or microscopic blood in the stool by Hemoccult testing. Clinical signs of Crohn's-like colitis are assessed using the Daily Activity Index (“DAI”). The DAI is the average of change in weight (0, <1%; 1, 1-5%; 2, 5-10%, 23, 10-20% and 4, >20%), intestinal bleeding (0, negative; 2, microscopic blood; 4, visible blood), and stool consistency (0, normal; 2, loose stools; 4, diarrhea).

b. Administration of Cyclophosphamide and/or a Cyclophosphamide Derivative in combination with GA

After establishment of Crohn's-like colitis (day 0), one half of the CD4+ CD45RB^{high} and one-half of the CD4+ CD45RB^{low} mice are administered cyclophosphamide and/or a cyclophosphamide derivative via intraperitoneal or intravenous injection in phosphate-buffered saline (PBS) (20 mg/ml) at a dose of 100-200 mg/kg. The cyclophosphamide derivatives include 4-hydroxycyclophosphamide, aldophosphamide, 4-hydroperoxycyclophosphamide, 4-(S-ethyl)cyclophosphamide and mafosfamide. On day 2 (±2), as determined by colitis progression), GA is administered subcutaneously at a dose of 500-2000 micrograms/mouse in PBS/mannitol for up to five consecutive days.

The other half of the CD4+ CD45RB^{high} and the CD4+ CD45RB^{low} mice are administered with mock injections (vehicle) as a control.

For the intravenous injections, the mice are warmed with a heat lamp (approximately 18-25 inches from the cage floor) while in their cage for 3-5 minutes to dilate their blood vessels; they are then individually restrained in a cone or Broome-type restraining device (VWR catalogue number 10718-030) for the intravenous injection administered into the lateral tail vein with a 28-30 gauge needle.

c. Results

Two months after treatment as described in step b, the mice are weighed and observed for clinical signs of Crohn's-like colitis, as described in step a above.

### Example 3

### Effect of Cyclophosphamide and/or Cyclophosphamide Derivatives in Combination with Glatiramer Acetate (“GA”) on Crohn's Disease in Mice (C3H.IL10−/− Model)

a. induction of Crohn's Colitis

Mice are randomized into treatment groups with the average body weight equivalent in each group. 6 to 12 week old C3H.IL10−/− mice are administered one oral gavage with 10⁸ LF82 E. coli bacteria to establish severe colitis.

After 2 months, the mice are weighed and observed for clinical signs of Crohn's-like colitis. Crohn's-like colitis is determined by weight loss, change in stool consistency, and/or microscopic blood in the stool by Hemoccult testing. Clinical signs of Crohn's-like colitis are assessed using the Daily Activity Index (“DAI”). The DAI is the average of change in weight (0, <1%; 1, 1-5%; 2, 5-10%, 23, 10-20% and 4, >20%), intestinal bleeding (0, negative; 2, microscopic blood; 4, visible blood), and stool consistency (0, normal; 2, loose stools; 4, diarrhea).

b. Administration of Cyclophosphamide and/or a Cyclophosphamide Derivative in Combination with GA

After establishment of Crohn's-like colitis (day 0), one-half of the mice are administered cyclophosphamide and/or a cyclophosphamide derivative via intraperitoneal or intravenous injection in phosphate-buffered saline (PBS) (20 mg/ml) at a dose of 100-200 mg/kg. The cyclophosphamide derivatives include 4-hydroxycyclophosphamide, aldophosphamide, 4-hydroperoxycyclophosphamide, 4-(S-ethyl)cyclophosphamide and mafosfamide. On day 2 (±2), as determined by colitis progression), GA is administered subcutaneously at a dose of 500-2000 micrograms/mouse in PBS/mannitol for up to five consecutive days.

The other half of the mice are administered with mock injections (vehicle) or intrarectal 50% ethanol as a control.

For the intravenous injections, the mice are warmed with a heat lamp (approximately 18-25 inches from the cage floor) while in their cage for 3-5 minutes to dilate their blood vessels; they are then individually restrained in a cone or Broome-type restraining device (VWR catalogue number 10718-030) for the intravenous injection administered into the lateral tail vein with a 28-30 gauge needle.

c. Results

Two months after treatment as described in step b, the mice are weighed and observed for clinical signs of Crohn's-like colitis, as described in step a above.

### Example 4

### Effect of Cyclophosphamide and/or Cyclophosphamide Derivatives in Combination with Glatiramer Acetate (“GA”) on Crohn's Disease in Humans

Human subjects are administered in an open-label format to with cyclophosphamide and/or a cyclophosphamide derivative and glatiramer acetate. The cyclophosphamide derivatives include 4-hydroxycyclophosphamide, aldophosphamide, 4-hydroperoxycyclophosphamide, 4-(S-ethyl)cyclophosphamide and mafosfamide. Human subjects are offered entry into the study if they meet all of the inclusion criteria and none of the exclusion criteria.

a. Inclusion/Exclusion Criteria

Human subjects are male or female, aged 18-70 inclusive. Human subjects must have evidence of ongoing disease activity with evidence of active disease on ileocolonoscopy and a Crohn's Disease of Activity Index of greater than 250.

Exclusion criteria are (1) any risk of pregnancy, (2) cardiac ejection fraction of <45%, (3) serum creatinine>2.0, (4) human subjects who are pre-terminal or moribund, (5) bilirubin>2.0, transaminases>2× normal, (5) human subjects with CDAI less than 250, (7) human subjects with active infections until infection is resolved or adequately managed, and (8) human subjects with WBC count<3000 cells/μL, platelets<100,000 cells/μL and untransfused hemoglobin<10 g/dL.

b. Administration of Cyclophosphamide and/or a Cyclophosphamide Derivative

Human subjects are administered cyclophosphamide or a cyclophosphamide derivative intravenously at a dose of 50 mg/kg/day on Day 3 to Day 0. The cyclophosphamide derivatives include 4-hydroxycyclophosphamide, aldophosphamide, 4-hydroperoxycyclophosphamide, 4-(S-ethyl)cyclophosphamide and mafosfamide.

Adequate diuresis should be maintained before and following cyclophosphamide derivative administration to prevent hemorrhagic cystitis. Prophylaxis for hemorrhagic cystitis (generally either MESNA (2-mercaptoethane sulfonate sodium) or forced diuresis) is directed according to established clinical practice guidelines used by the SCT (stem cell transplant) program.

On Day 6 (six days after the last dose of cyclophosphamide derivative) all human subjects receive GCSF at a dose of 5 μg/kg/day until their absolute neutrophil count exceeds 1.0×10⁹ per liter for two consecutive days. Human subjects are also routinely given antibiotics (norfloxacin, fluconazole or valacyclovir) until their absolute neutrophil count exceeds 1.0×10⁹ per liter for two consecutive days.

c. Administration of Glatiramer Acetate

Human subjects are administered with glatiramer acetate subcutaneously at a dose of 20 mg/kg on Day 30 (30 days after the last dose of cyclophosphamide or cyclophosphamide derivative), and continue to receive daily doses of glatiramer acetate at 20 mg/kg indefinitely.

d. Evaluation of Results

Baseline clinical evaluations are conducted at months −3 and 0; treatment and follow-up visits at months 3, 6, 9, 12, 15, 18, 21 and 24 months. Colonoscopies are conducted at months −3,3,12, and 24 to monitor the course of the disease progression after treatment.

### Example 5

### Mouse Model for Treatment of MS with a Cyclophosphamide Derivative and Glatiramer Acetate

a. Overview

Chronic relapsing experimental autoimmune encephalomyelitis (R-EAE) is a well-established non-primate animal model for relapsing/remitting human MS (“RRMS”). R-EAE is induced by immunization of susceptible mouse strains (SJL/J) with modified myelin proteolipid protein (PLP)139-151 peptide (HSLGKWLGHPDKF). R-EAE takes approximately takes 2-3 weeks to develop. Treatment groups consist of 10 mice.

b. R-EAE Induction and Analysis

Female SJL/J mice between 6-12 weeks old receive one subcutaneous 100 microliter injection with 100 micrograms of modified myelin proteolipid protein (PLP)139-151 peptide (HSLGKWLGHPDKF) that is emulsified in Freund's Incomplete Adjuvant containing Mycobacterium tuberculisis H37 Ra (CFA). CFA is an oil mixture of Freund's Incomplete Adjuvant and heat-killed Mycobacterium tuberculosis. For the subcutaneous adjuvant injection, the animal is held by the loose skin at the nape of its neck and injected with a 25 gauge needle in the thigh area.

Mice are weighed and observed for clinical signs for 2 months.

Clinical signs of EAE are assessed according to the following scale: 0=no clinical disease; 1=loss of tail tonicity; 2=mild hind leg paresis; 3=moderate hind leg paralysis; 4=complete paraplegia; and 5=quadriplegia, moribund state or death. Additional behavioral outcomes may be monitored including: the animals' ability to lift their tail while walking or when touched, their ability to move all four limbs and walk in a coordinated way along the rungs of the cage lid, and overall activity level and exploratory tendencies.

EAE in the SJL/J mouse strain is an escalating type of paralysis where symptoms are preceded by obvious weight loss and mild paresis of the tail by day 10. At later time points, disease progression involves hind limb paralysis, which constitutes the effector phase, first attack of the R-EAE disease phenotype. By day 20, most mice enter the remission phase, regain weight and paralysis is abated.

Mice develop a second round of EAE (relapse) at approximately 3 weeks post PLP-immunization and mice will continue to experience additional relapses and recovery phases.

Sensory function is also determined using a thermal sensory test. This involves determining whether there is sensation in the extremities by setting the mouse on a platform that heats up; although the heat is not extreme to the point of injury, the expected response is that the mouse should lift and lick its paw.

c. Administration of Combination Therapy

The cyclophosphamide derivatives used in the study include 4-hydroxycyclophosphamide, aldophosphamide, 4-hydroperoxycyclophosphamide, 4-(S-ethyl)cyclophosphamide and mafosfamide. A cyclophosphamide derivative is administered via intraperitoneal injection in phosphate-buffered saline (PBS) (20 mg/ml) at a dose of 100-300 mg/kg. The cyclophosphamide derivative is administered once to each study animal at specific time points prior to and after the effector phase of R-EAE.

Glatiramer acetate is administered subcutaneously at a dose of 50-500 micrograms/mouse in PBS/mannitol for up to five consecutive days. For the intravenous injections, the mice are warmed while in their cage with a heat lamp (approximately 18-25 inches from the cage floor) for 3-5 minutes to dilate their blood vessels; they are then individually restrained in a cone or Broome-type restraining device (VWR catalogue number 10718-030) for the intravenous injection administered into the lateral tail vein with a 28-30 gauge needle. Light isofurane anesthesia can be used.

Food is placed on the cage floor to help injured animals reach food easily. The automatic watering system is lower to the cage floor than a water bottle would be; however, if sick animals don't appear to have the strength to operate the water dispensing switch, a water bottle (and possibly direct feeding of water to individual animals at the time of their daily weigh-iris) and/or ‘hydrogel’ is made available to the animals in those cages.

### Example 6

### Mouse Model for Treatment of MS with a Cyclophosphamide Derivative in Combination with ATG

a. Overview

Chronic relapsing experimental autoimmune encephalomyelitis (R-EAE) is a well-established non-primate animal model for relapsing/remitting human MS (“RRMS”). R-EAE is induced by immunization of susceptible mouse strains (SJL/J) with modified myelin proteolipid protein (PLP)139-151 peptide (HSLGKWLGHPDKF). R-EAE takes approximately takes 2-3 weeks to develop. Treatment groups consist of 10 mice.

b. R-EAE Induction and Analysis

Female SJL/J mice between 6-12 weeks old receive one subcutaneous 100 microliter injection with 100 micrograms of modified myelin proteolipid protein (PLP)139-151 peptide (HSLGKWLGHPDKF) that is emulsified in Freund's Incomplete Adjuvant containing Mycobacterium tuberculisis H37 Ra(CFA). CFA is an oil mixture of Freund's Incomplete Adjuvant and heat-killed Mycobacterium tuberculosis. For the subcutaneous adjuvant injection, the animal is held by the loose skin at the nape of its neck and injected with a 25 gauge needle in the thigh area.

Mice are weighed and observed for clinical sighs for 2 months.

Clinical signs of EAE are assessed according to the following scale: 0=no clinical disease; 1=loss of tail tonicity; 2=mild hind leg paresis; 3=moderate hind leg paralysis; 4=complete paraplegia; and 5=quadriplegia, moribund state or death. Additional behavioral outcomes may be monitored including: the animals' ability to lift their tail while walking or when touched, their ability to move all four limbs and walk in a coordinated way along the rungs of the cage lid, and overall activity level and exploratory tendencies.

EAE in the SJL/J mouse strain is an escalating type of paralysis where symptoms are preceded by obvious weight loss and mild paresis of the tail by day 10. At later time points, disease progression involves hind limb paralysis, which constitutes the effector phase, first attack of the R-EAE disease phenotype. By day 20, most mice enter the remission phase, regain weight and paralysis is abated.

Mice develop a second round of EAE (relapse) at approximately 3 weeks post PLP-immunization and mice will continue to experience additional relapses and recovery phases.

Sensory function is also determined using a thermal sensory test. This involves determining whether there is sensation in the extremities by setting the mouse on a platform that heats up; although the heat is not extreme to the point of injury, the expected response is that the mouse should lift and lick its paw.

c. Administration of Combination Therapy

The cyclophosphamide derivatives used in the study include 4-hydroxycyclophosphamide, aldophosphamide, 4-hydroperoxycyclophosphamide, 4-(S-ethyl)cyclophosphamide and mafosfamide. A cyclophosphamide derivative is administered via intraperitoneal injection in phosphate-buffered saline (PBS) (20 mg/ml) at a dose of 100-300 mg/kg. Cyclophosphamide derivative is administered once to each study animal at specific time points prior to and after the effector phase of R-EAE. Antithymocyte antibodies raised against whole T lymphocytes are administered intravenously concurrently with cyclophosphamide derivative at doses ranging from 12.5 to 25 micrograms per animal.

### Example 7

### Clinical Trial of a Cyclophosphamide Derivative and Glatiramer Acetate in Humans Diagnosed with Multiple Sclerosis

a. Overview

A 12-month, randomized, multi-center, rater-blinded (pre and post) trial in approximately 222 individuals with aggressive relapsing remitting MS (RRMS) with a follow up extension of 12 months is conducted. One of the following cyclophosphamide derivatives is selected for the trial: 4-hydroxycyclophosphamide, aldophosphamide, 4-hydroperoxycyclophosphamide, 4-(S-ethyl)cyclophosphamide and mafosfamide. Approximately 222 individuals are randomized to one of the following three treatment arms (74 in each arm): a.) low dose the cyclophosphamide derivative and double dose glatiramer acetate; b.) high dose the cyclophosphamide derivative; and c.) high dose the cyclophosphamide derivative and double dose glatiramer acetate.

i. Inclusion Criteria

Individuals, male or female, meeting all of the following criteria are enrolled in the clinical trial: a.) between the ages of 18 and 50 years; b.) a diagnosis of clinically definite relapsing-remitting MS according to the McDonald Criteria; c.) two (2) or more total gadolinium enhancing lesions on a brain and/or spinal cord MRI at screening; d.) at least one clinical relapse in the last year; e.) an EDSS ranging from 1.5 to 6.5 inclusive (individuals with EDSS>5.5 should have been sustained at that disability for <3 months); f.) a sustained (>3 months) increase of >1.0 on the EDSS (historical estimate allowed) between 1.5 and 5.5 or >0.5 between 5.5 and 6.5 in the preceding year; g.) written informed consent prior to any testing under this protocol, including screening tests and evaluations that are not considered part of the individual's routine care; and h.) for females, a negative pregnancy test prior to entry into the study.

ii. Exclusion Criteria

The following individuals are excluded from the clinical trial: a.) any individual at risk of pregnancy; b.) any individual exhibiting cardiac ejection fraction of <45%; c.) any individual exhibiting serum creatinine levels>2.0; d.) any individual who is pre-terminal or moribund; e.) any individual exhibiting bilirubin levels>2.0, and/or transaminases levels>2× normal; f.) any individual with pacemakers and implants who cannot get serial MRIs; g.) any individual with active infections until infection is resolved; or h.) any individual with WBC count<3000 cells/μL; platelets<100,000 cells/μL; and untransfused hemoglobin<100 g/dL.

iii. Removal of Individuals from the Study

Individuals may withdraw from the study at any time for any reason.

Any investigator may discontinue a individual for any of the following reasons: a.) the individual experiences a medical emergency that necessitates discontinuation of therapy during the high dose the cyclophosphamide derivative treatment in the hospital; b.) the individual experiences a serious adverse event that is judged to be likely related to the high dose of the cyclophosphamide derivative and/or is of severity that warrants discontinuation of high dose of the cyclophosphamide derivative during hospital stay; and c.) for any medical reason at the discretion of the investigator.

c. Administration of Combination Therapy

i. High-Dose Cyclophosphamide Derivative Administration

Cyclophosphamide derivative treatment is performed under the supervision of Oncology physicians and staff.

Individuals receive the cyclophosphamide derivative intravenously on Day −3 to Day 0. The dose of the cyclophosphamide derivative is calculated according to ideal body weight. Ideal body weight is determined according to the current policy used in the Bone Marrow Transplant program. If the individual's actual weight is less than ideal, the actual weight is used to calculate the dose of the cyclophosphamide derivative. Individuals are scheduled to receive only one course of therapy.

Adequate diuresis is maintained before and following cyclophosphamide derivative administration to prevent hemorrhagic cystitis. Prophylaxis for hemorrhagic cystitis (generally either MESNA or forced diuresis) is directed according to established clinical practice guidelines used by the SCT program.

On Day 6 individuals receive granulocyte colony stimulating factor (5 mg/kg/d) until the absolute neutrophil count exceeds 1.0×10⁹ per liter for two consecutive days. Individuals are also given antibiotics (norfloxacin, fluconazole and valacyclovir) until the return of normal neutrophil counts.

ii. Low Dose Cyclophosphamide Derivative Administration

The cyclophosphamide derivative is administered at 1000 mg/m² FV in 100 cc NSS over two hours. Prehydration will consist of 2L NSS over 4 hours and post-hydration will consist of 2L NSS over 4 hours. Dose will be calculated according to ideal body weight as above.

iii. Glatiramer Acetate Administration

Double dose glatiramer acetate is administered daily subcutaneously beginning at 30 days after the last dose of high dose cyclophosphamide derivative (Day 0) or the single lower dose cyclophosphamide derivative injection.

iv. Post Treatment Discharge

Individuals are hospitalized for a minimum of 4 days as clinically indicated. They are then admitted to an out individual care facility until return of neutrophil count as per standard protocols (usually 2-3 weeks after the last dose of high dose cyclophosphamide derivative).

c. Evaluation of Results

i. MRI Evaluations

MRI evaluations are conducted at months −3, 0, 3, 6, 9, 12, 15, 18, 21 and 24 after treatment. The mean number of gadolinium enhancing lesions is monitored to assess the change in disease activity. Change from baseline (average number of gad-enhancing lesions at months −3 and 0) to follow-up (average number of gad-enhancing lesions at months 15 and 18) is assessed. Other parameters—T2 lesion load and brain parenchymal fraction are also measures of disease activity that correlate with accrual of disability and changes will be assessed through the length of the study. Scans are performed on a 1.5 Tesla General Electric scanner (Milwaukee Wis.) with echo speed or twin speed gradients.

MRI criteria for disease progression: a.) number of gadolinium enhancing lesions; b.) T2 lesion load; and c.) brain parenchymal fraction.

Analysis of MRI Scans:

a.) Contrast-enhancing lesions will be counted from the axial 3 mm contiguous slices with verification on the coronal images. If a lesion is seen on one sequence but not the other, it is counted as an enhancing plaque if it is also seen on a long TR pulse sequence. Total disease burden is determined from scans from the cervicomedullary junction to the vertex based on the number of enhancing plaques.

b.) The volume of multiple sclerosis plaques is determined from analysis of the FLAIR scans as they provide the maximal contrast to noise between MS plaques and underlying cerebrospinal fluid (CSF) versus normal white and gray matter. However in the event of cystic MS plaques which would have dark signal on FLAIR scans, the proton density-T2-weighted pulse sequences are utilized to identify these lesions and supplement the FLAIR volume assessment with these additional MS plaques. Thresholding and 3D volumetric analysis is performed using computer-assisted volumetry.

c.) Total brain parenchymal volume is performed suing standard stripping algorithms to remove the skull and overlying soft tissue. Using thresholding and manual corrections, the CSF is then removed to allow an analysis of brain parenchyma volume.

d.) Two radiologists read the MRI scans independently. If there is greater than 10% discrepancy between interpretations, a third radiologist is asked to interpret the MRI scans. The reported interpretation is the average of the three readings (on T2 plaque volume and brain parenchymal fraction) or reflects the two interpretations in agreement (for the number of enhancing lesions). The data is recorded on the CRPs and input into the database.

ii. Neurological/Clinical Evaluation

A neurological exam is conducted at baseline and every 3 months after the high dose cyclophosphamide derivative treatment for the duration of the study (24 months). To determine the course of the disease, the clinical measures used are the Multiple Sclerosis Functional Composite (MSFC) and the Expanded Disability Status Scale (EDSS). A research nurse/coordinator is trained to administer the MSFC and a study neurologist examines the individual to provide an EDSS score.

The EDSS ranges from 0 (normal) to 10 (death due to MS), based on neurological examination of eight functional systems (visual, brainstem, sensory, cerebellar, sphincter, cerebral and others).

The MSFC is designed to test gait, upper extremity dexterity and cognition. The three subtests are (a) 25 foot timed walk (25TW); (b) 9-hole peg test (9-HPT); and (c) Paced Auditory Serial Addition Test (PASAT-3). The PASAT test requires individuals to add consecutive numbers as they are presented on an auditory tape and respond orally with the accurate sum. As each digit is presented, the individual must sum that number with the digit that was presented prior to it rather than with the individual's previous response.

### Example 8

### Clinical Trial of a Cyclophosphamide Derivative and ATG in Humans Diagnosed with Multiple Sclerosis

a. Overview

A 12-month, randomized, multi-center, rater-blinded (pre and post) trial in approximately 222 individuals with aggressive relapsing remitting MS (RRMS) with a follow up extension of 12 months is conducted. One of the following cyclophosphamide derivatives is selected for the trial: 4-hydroxycyclophosphamide, aldophosphamide, 4-hydroperoxycyclophosphamide, 4-(S-ethyl)cyclophosphamide and mafosfamide. Approximately 222 individuals will be randomized to one of the following two treatment arms (111 in each arm): a.) high dose cyclophosphamide derivative; and b.) high dose cyclophosphamide derivative and antithymocyte globulin.

i. Inclusion Criteria

Individuals, male or female, meeting all of the following criteria are enrolled in the clinical trial: a.) between the ages of 18 and 50 years; b.) a diagnosis of clinically definite relapsing-remitting MS according to the McDonald Criteria; c.) two (2) or more total gadolinium enhancing lesions on a brain and/or spinal cord MRI at screening; d.) at least one clinical relapse in the last year; e.) an EDSS ranging from 1.5 to 6.5 inclusive (individuals with EDSS>5.5 should have been sustained at that disability for <3 months); f.) a sustained (>3 months) increase of >1.0 on the EDSS (historical estimate allowed) between 1.5 and 5.5 or >0.5 between 5.5 and 6.5 in the preceding year; g.) written informed consent prior to any testing under this protocol, including screening tests and evaluations that are not considered part of the individual's routine care; and h.) for females, a negative pregnancy test prior to entry into the study.

ii. Exclusion Criteria

The following individuals are excluded from the clinical trial: a.) any individual at risk of pregnancy; b.) any individual exhibiting cardiac ejection fraction of <45%; c.) any individual exhibiting serum creatinine levels>2.0; d.) any individual who is pre-terminal or moribund; c.) any individual exhibiting bilirubin levels>2.0, and/or transaminases levels>2× normal; f.) any individual with pacemakers and implants who cannot get serial MRIs; g.) any individual with active infections until infection is resolved; or h.) any individual with WBC count<3000 cells/μL; platelets<100,000 cells/μL; and untransfused hemoglobin<10 g/dL.

iii. Removal of Individuals from the Study

Individuals may withdraw from the study at any time for any reason.

Any investigator may discontinue a individual for any of the following reasons: a.) the individual experiences a medical emergency that necessitates discontinuation of therapy during the high dose cyclophosphamide derivative treatment in the hospital; b.) the individual experiences a serious adverse event that is judged to be likely related to high dose cyclophosphamide derivative and/or is of severity that warrants discontinuation of high dose cyclophosphamide derivative during hospital stay; and c.) for any medical reason at the discretion of the investigator.

b. Administration of Combination Therapy

i. High Dose Cyclophosphamide Derivative Administration

Cyclophosphamide derivative treatment is performed under the supervision of Oncology physicians and staff.

Individuals receive the cyclophosphamide derivative 50 mg/kg/d intravenously on Day −3 to Day 0. The dose of the cyclophosphamide derivative will be calculated according to ideal body weight. Ideal body weight is determined according to the current policy used in the Bone Marrow Transplant program. If the individual's actual weight is less than ideal, the actual weight is used to calculate the dose of the cyclophosphamide derivative. Individuals are scheduled to receive only one course of therapy.

Adequate diuresis is maintained before and following cyclophosphamide derivative administration to prevent hemorrhagic cystitis. Prophylaxis for hemorrhagic cystitis (generally either MESNA or forced diuresis) is directed according to established clinical practice guidelines used by the SCT program.

On Day 6 (six days after completion of the cyclophosphamide derivative) individuals receive granulocyte colony stimulating factor (5 μg/kg/d) until the absolute neutrophil count exceeds 1.0×10⁹ per liter for two consecutive days. Individuals are also given antibiotics (norfloxacin, fluconazole and valacyclovir) until the return of normal neutrophil counts.

ii. Antithymocyte Globulin Administration

Antithymocyte globulin is administered daily by IV concurrently with high dose the cyclophosphamide derivative.

iii. Post Treatment Discharge

Individuals are hospitalized for a minimum of 4 days as clinically indicated. They are then admitted to an out individual care facility until return of neutrophil count as per standard protocols (usually 2-3 weeks after the last dose of high dose the cyclophosphamide derivative).

c. Evaluation of Results

i. MRI Evaluations

MRI evaluations are conducted at months −3, 0, 3, 6, 9, 12, 15, 18, 21 and 24 after treatment. The mean number of gadolinium enhancing lesions is monitored to assess the change in disease activity. Change from baseline (average number of gad-enhancing lesions at months −3 and 0) to follow-up (average number of gad-enhancing lesions at months 15 and 18) is assessed. Other parameters—T2 lesion load and brain parenchymal fraction are also measures of disease activity that correlate with accrual of disability and changes will be assessed through the length of the study. Scans are performed on a 1.5 Tesla General Electric scanner (Milwaukee Wis.) with echo speed or twin speed gradients.

MRI criteria for disease progression: a.) number of gadolinium enhancing lesions; b.) T2 lesion load; and c.) brain parenchymal fraction.

Analysis of MRI Scans:

a.) Contrast-enhancing lesions will be counted from the axial 3 mm contiguous slices with verification on the coronal images. If a lesion is seen on one sequence but not the other, it is counted as an enhancing plaque if it is also seen on a long TR pulse sequence. Total disease burden is determined from scans from the cervicomedullary junction to the vertex based on the number of enhancing plaques.

b.) The volume of multiple sclerosis plaques is determined from analysis of the FLAIR scans as they provide the maximal contrast to noise between MS plaques and underlying cerebrospinal fluid (CSF) versus normal white and gray matter. However in the event of cystic MS plaques which would have dark signal on FLAIR scans, the proton density-T2-weighted pulse sequences are utilized to identify these lesions and supplement the FLAIR volume assessment with these additional MS plaques. Thresholding and 3D volumetric analysis is performed using computer-assisted volumetry.

c.) Total brain parenchymal volume is performed suing standard stripping algorithms to remove the skull and overlying soft tissue. Using thresholding and manual corrections, the CSF is then removed to allow an analysis of brain parenchyma volume.

d.) Two radiologists read the MRI scans independently. If there is greater than 10% discrepancy between interpretations, a third radiologist is asked to interpret the MRI scans. The reported interpretation is the average of the three readings (on T2 plaque volume and brain parenchymal fraction) or reflects the two interpretations in agreement (for the number of enhancing lesions). The data is recorded on the CRPs and input into the database.

ii. Neurological/Clinical Evaluation

A neurological exam is conducted at baseline and every 3 months after the high dose cyclophosphamide derivative treatment for the duration of the study (24 months). To determine the course of the disease, the clinical measures used are the Multiple Sclerosis Functional Composite (MSFC) and the Expanded Disability Status Scale (EDSS). A research nurse/coordinator is trained to administer the MSFC and a study neurologist examines the individual to provide an EDSS score.

The EDSS ranges from 0 (normal) to 10 (death due to MS), based on neurological examination of eight functional systems (visual, brainstem, sensory, cerebellar, sphincter, cerebral and others).

The MSFC is designed to test gait, upper extremity dexterity and cognition. The three subtests are (a) 25 foot timed walk (25TW); (b) 9-hole peg test (9-HPT); and (c) Paced Auditory Serial Addition Test (PASAT-3). The PASAT test requires individuals to add consecutive numbers as they are presented on an auditory tape and respond orally with the accurate sum. As each digit is presented, the individual must sum that number with the digit that was presented prior to it rather than with the individual's previous response.

### Example 9

### Clinical Trial of a Cyclophosphamide Derivative in Combination with ATG, and Glatiramer Acetate in Humans Diagnosed with Multiple Sclerosis

a. Overview

A 12-month, randomized, multi-center, rater-blinded (pre and post) trial in approximately 222 individuals with aggressive relapsing remitting MS (RRMS) with a follow up extension of 12 months is conducted. One of the following cyclophosphamide derivatives is selected for the trial: 4-hydroxycyclophosphamide, aldophosphamide, 4-hydroperoxycyclophosphamide, 4-(S-ethyl)cyclophosphamide and mafosfamide. Approximately 222 individuals will be randomized to one of the following two treatment arms (111 in each arm): a.) high dose cyclophosphamide derivative; and b.) high dose cyclophosphamide derivative, antithymocyte globulin, and glatiramer acetate.

i. Inclusion Criteria

Individuals, male or female, meeting all of the following criteria are enrolled in the clinical trial: a.) between the ages of 18 and 50 years; b.) a diagnosis of clinically definite relapsing-remitting MS according to the McDonald Criteria; c.) two (2) or more total gadolinium enhancing lesions on a brain and/or spinal cord MRI at screening; d.) at least one clinical relapse in the last year; e.) an EDSS ranging from 1.5 to 6.5 inclusive (individuals with EDSS >5.5 should have been sustained at that disability for <3 months); f.) a sustained (>3 months) increase of >1.0 on the EDSS (historical estimate allowed) between 1.5 and 5.5 or >0.5 between 5.5 and 6.5 in the preceding year; g.) written informed consent prior to any testing under this protocol, including screening tests and evaluations that are not considered part of the individual's routine care; and h.) for females, a negative pregnancy test prior to entry into the study.

ii. Exclusion Criteria

The following individuals are excluded from the clinical trial: a.) any individual at risk of pregnancy; b.) any individual exhibiting cardiac ejection fraction of <45%; c.) any individual exhibiting serum creatinine levels>2.0; d.) any individual who is pre-terminal or moribund; e.) any individual exhibiting bilirubin levels>2.0, and/or transaminases levels>2× normal; f.) any individual with pacemakers and implants who cannot get serial MRIs; g.) any individual with active infections until infection is resolved; or h.) any individual with WBC count<3000 cells/μL; platelets<100,000 cells/μL; and untransfused hemoglobin<10 g/dL.

iii. Removal of Individuals from the Study

Individuals may withdraw from the study at any time for any reason.

Any investigator may discontinue an individual for any of the following reasons: a.) the individual experiences a medical emergency that necessitates discontinuation of therapy during the high dose cyclophosphamide derivative treatment in the hospital; b.) the individual experiences a serious adverse event that is judged to be likely related to high dose cyclophosphamide derivative and/or is of severity that warrants discontinuation of high dose cyclophosphamide derivative during hospital stay; and c.) for any medical reason at the discretion of the investigator.

b. Administration of Combination Therapy

i. High Dose Cyclophosphamide Derivative Administration

Cyclophosphamide derivative treatment is performed under the supervision of Oncology physicians and staff.

Individuals receive the cyclophosphamide derivative 50 mg/kg/d intravenously on Day −3 to Day 0. The dose of cyclophosphamide derivative will be calculated according to ideal body weight. Ideal body weight is determined according to the current policy used in the Bone Marrow Transplant program. If the individual's actual weight is less than ideal, the actual weight is used to calculate the dose of cyclophosphamide derivative. Individuals are scheduled to receive only one course of therapy.

Adequate diuresis is maintained before and following cyclophosphamide derivative administration to prevent hemorrhagic cystitis. Prophylaxis for hemorrhagic cystitis (generally either MESNA or forced diuresis) is directed according to established clinical practice guidelines used by the SCT program.

On Day 6 (six days after completion of cyclophosphamide derivative) individuals receive granulocyte colony stimulating factor (5 μg/kg/d) until the absolute neutrophil count exceeds 1.0×10⁹ per liter for two consecutive days. Individuals are also given antibiotics (norfloxacin, fluconazole and valacyclovir) until the return of normal neutrophil counts.

ii. Antithymocyte Globulin Administration

Antithymocyte globulin is administered daily by IV concurrently with high dose cyclophosphamide derivative.

iii. Glatiramer Acetate Administration

Double dose glatiramer acetate will be administered daily subcutaneously beginning at 30 days after the last dose of high dose cyclophosphamide derivative (Day 0).

iv. Post Treatment Discharge

Individuals are hospitalized for a minimum of 4 days as clinically indicated. They are then admitted to an out individual care facility until return of neutrophil count as per standard protocols (usually 2-3 weeks after the last dose of high dose cyclophosphamide derivative).

c. Evaluation of Results

i. MRI Evaluations

MRI evaluations are conducted at months −3, 0, 3, 6, 9, 12, 15, 18, 21 and 24 after treatment. The mean number of gadolinium enhancing lesions is monitored to assess the change in disease activity. Change from baseline (average number of gad-enhancing lesions at months −3 and 0) to follow-up (average number of gad-enhancing lesions at months 15 and 18) is assessed. Other parameters—T2 lesion load and brain parenchymal fraction are also measures of disease activity that correlate with accrual of disability and changes will be assessed through the length of the study. Scans are performed on a 1.5 Tesla General Electric scanner (Milwaukee Wis.) with echo speed or twin speed gradients.

MRI criteria for disease progression: a.) number of gadolinium enhancing lesions; b.) T2 lesion load; and c.) brain parenchymal fraction.

Analysis of MRI Scans:

a.) Contrast-enhancing lesions will be counted from the axial 3 mm contiguous slices with verification on the coronal images. If a lesion is seen on one sequence but not the other, it is counted as an enhancing plaque if it is also seen on a long TR pulse sequence. Total disease burden is determined from scans from the cervicomedullary junction to the vertex based on the number of enhancing plaques.

b.) The volume of multiple sclerosis plaques is determined from analysis of the FLAIR scans as they provide the maximal contrast to noise between MS plaques and underlying cerebrospinal fluid (CSF) versus normal white and gray matter. However in the event of cystic MS plaques which would have dark signal on FLAIR scans, the proton density-T2-weighted pulse sequences are utilized to identify these lesions and supplement the FLAIR volume assessment with these additional MS plaques. Thresholding and 3D volumetric analysis is performed using computer-assisted volumetry.

c.) Total brain parenchymal volume is performed using standard stripping algorithms to remove the skull and overlying soft tissue. Using thresholding and manual corrections, the CSF is then removed to allow an analysis of brain parenchyma volume.

d.) Two radiologists read the MRI scans independently. If there is greater than 10% discrepancy between interpretations, a third radiologist is asked to interpret the MRI scans. The reported interpretation is the average of the three readings (on T2 plaque volume and brain parenchymal fraction) or reflects the two interpretations in agreement (for the number of enhancing lesions). The data is recorded on the CRPs and input into the database.

ii. Neurological/Clinical Evaluation

A neurological exam is conducted at baseline and every 3 months after the high dose cyclophosphamide derivative treatment for the duration of the study (24 months). To determine the course of the disease, the clinical measures used are the Multiple Sclerosis Functional Composite (MSFC) and the Expanded Disability Status Scale (EDSS). A research nurse/coordinator is trained to administer the MSFC and a study neurologist examines the individual to provide an EDSS score.

The EDSS ranges from 0 (normal) to 10 (death due to MS), based on neurological examination of eight functional systems (visual, brainstem, sensory, cerebellar, sphincter, cerebral and others).

The MSFC is designed to test gait, upper extremity dexterity and cognition. The three subtests are (a) 25 foot timed walk (25TW); (b) 9-hole peg test (9-HPT); and (c) Paced Auditory Serial Addition Test (PASAT-3). The PASAT test requires individuals to add consecutive numbers as they are presented on an auditory tape and respond orally with the accurate sum. As each digit is presented, the individual must sum that number with the digit that was presented prior to it rather than with the individual's previous response.

1. A method for treating or preventing an autoimmune disease, the method comprising administering to a subject in need thereof:
a) a lymphablative agent selected from the group consisting of cyclophosphamide and a cyclophosphamide derivative; and
b) an additional immunomodulatory agent selected from the group consisting of an amino acid co-polymer, a peptide fragment of myelin basic protein, an anti-tumor necrosis factor agent and pharmaceutically acceptable salts thereof;
wherein the autoimmune disease selected from the group consisting of inflammatory bowel disease, rheumatoid arthritis, diabetes mellitus, celiac disease, autoimmune thyroid disease, autoimmune liver disease, Addison's Disease, Sjögren's Syndrome, transplant rejection, graft vs. host disease and host vs. graft disease.

2. (canceled)

3. The method of claim 1, wherein the lymphablative agent is a cyclophosphamide derivative selected from the group consisting of: 4-hydroperoxycyclophosphamide, mafosfamide, 4-hydroxycyclophosphamide, aldophosphamide and 4-(S-alkyl)cyclophosphamide.

4. The method of claim 1, wherein the autoimmune disease is inflammatory bowel disease selected from the group consisting of inflammatory bowel disease, rheumatoid arthritis, diabetes mellitus, celiac disease, autoimmune thyroid disease, autoimmune liver disease, Addison's Disease, Sjögren's Syndrome, transplant rejection, graft vs. host disease, host vs. graft disease, multiple sclerosis, Guillain-Barre syndrome, Lambert-Eaton myasthenic syndrome, myasthenia gravis, transverse myelitis, systemic lupus erythematosus (SLE or lupus), acute disseminated encephalomyelitis, autoimmune inner ear disease, narcolepsy, neuromyotonia, or schizophrenia.

5.-6. (canceled)

7. The method of claim 1, wherein the lymphablative agent is administered at an amount effective to achieve immune lymphablation in the subject.

8.-11. (canceled)

12. The method of claim 1, further comprising administering to the subject an anti-lymphocyte antibody.

13. The method of claim 12, wherein the anti-lymphocyte antibody is administered in an amount that is effective to reduce the number of T cells in the subject.

14. (canceled)

15. The method of claim 12, wherein the anti-lymphocyte antibody is administered concurrently with or subsequent to the administration of the lymphablative agent.

16. (canceled)

17. The method of claim 12, wherein the anti-lymphocyte antibody is anti-CD3 antibody, anti-CD4 antibody, anti-CD8 antibody, anti-lymphocyte serum, an anti-natural killer cell antibody, or an anti-CD40L antibody.

18. The method of claim 1, wherein the additional immunomodulatory agent is an amino acid co-polymer or a pharmaceutically acceptable salt thereof.

19. The method of claim 18, wherein the amino acid co-polymer is a YEAK, VWAK, FYAK, YFAK, or VYAK co-polymer.

20.-25. (canceled)

26. The method of claim 1, wherein the additional immunomodulatory agent is a peptide fragment of myelin basic protein or a pharmaceutically acceptable salt thereof.

27. The method of claim 26, wherein the peptide fragment of myelin basic protein is:
(SEQ ID NO: 2)
Asp-Glu-Asn-Pro-Val-Val-His-Phe-Phe-Lys-Asn-Ile-
Val-Thr-Pro-Arg-Thr;
(SEQ ID NO: 3)
Lys-Ser-His-Gly-Arg-Thr-Gln-Asp-Glu-Asn-Pro-Val-
Val-His-Phe-Phe-Lys-Asn-Ile-Val-Thr;
(SEQ ID NO: 4)
Ala-Arg-Thr-Ala-His-Tyr-Gly-Ser-Leu-Pro-Gln-Lys-
Ser-His-Gly;
(SEQ ID NO: 5)
His-His-Pro-Ala-Arg-Thr-Ala-His-Tyr-Gly-Ser-Leu-
Pro-Gln-Lys;
(SEQ ID NO: 6)
Tyr-Gly-Ser-Leu-Pro-Gln-Lys-Ser-His-Gly-Arg-Thr-
Gln-Asp-Glu;
(SEQ ID NO: 7)
Thr-Gln-Asp-Glu-Asn-Pro-Val-Val-His-Phe-Phe-Lys-
Asn-Ile-Val-Thr-Pro-Arg;
(SEQ ID NO: 8)
Lys-Asn-Ile-Val-Thr-Pro-Arg-Thr-Pro-Pro-Pro-Ser-
Gln-Gly-Lys-Gly;
(SEQ ID NO: 9)
Asn-Pro-Val-Val-His-Phe-Phe-Lys-Asn-Ile;
(SEQ ID NO: 10)
Pro-Val-Val-His-Phe-Phe-Lys-Asn-Ile-Val;
(SEQ ID NO: 11)
Val-Val-His-Phe-Phe-Lys-Asn-Ile-Val-Thr;
or
(SEQ ID NO: 12)
Val-His-Phe-Phe-Lys-Asn-Ile-Val-Thr-Pro.

28.-32. (canceled)

33. The method of claim 1, wherein the additional immunomodulatory agent is an anti-tumor necrosis factor agent.

34. The method of claim 33, wherein the anti-tumor necrosis factor agent is infliximab, adalimumab, certolizumab, or golimumab.

35.-38. (canceled)

39. The method of claim 1, further comprising administering an effective amount of granulocyte colony stimulating factor.

40. The method of claim 1, further comprising administering an effective amount of an antibiotic.

41.-64. (canceled)

65. The method of claim 1, further comprising administering vitamin D to the subject.

66.-141. (canceled)

142. A composition comprising (a) an effective amount of cyclophosphamide or a cyclophosphamide derivative; and (b) an additional immunomodulatory agent selected from the group consisting of an amino acid co-polymer, a peptide fragment of myelin basic protein, an anti-tumor necrosis factor agent, an anti-lymphocyte antibody and pharmaceutically acceptable salts, solvates and isomers thereof, in an effective amount to prevent or treat an autoimmune disease in a subject.

143. The composition of claim 142, wherein the composition comprises a cyclophosphamide derivative selected from the group consisting of 4-hydroperoxycyclophosphamide, mafosfamide, 4-hydroxycyclophosphamide, aldophosphamide and 4-(S-alkyl)cyclophosphamide.

144. (canceled)

145. The composition of claim 142, further comprising administering to the subject an anti-lymphocyte antibody.
